(19) United States
(12) Patent Application Publication
(10) Pub. No.: US 2022/0257523 A1
(43) Pub. Date: Aug. 18, 2022

(54) COMPOSITE DRUG PARTICLES AND USES THEREOF

(71) Applicant: The Regents of the University of Michigan, Ann Arbor, MI (US)

(72) Inventors: Michael L. FELDER, Ann Arbor, MI (US); Omolola ENIOLA-ADEFESO, Ann Arbor, MI (US); Hanieh SAFARI, Ann Arbor, MI (US)

(73) Assignee: The Regents of the University of Michigan, Ann Arbor, MI (US)

(21) Appl. No.: 17/627,571

(22) PCT Filed: Jul. 16, 2020
(86) PCT No.: PCT/US2020/042293

§ 371 Date: Jan. 14, 2022

Related U.S. Application Data
(60) Provisional application No. 62/874,784, filed on Jul. 16, 2019.

(51) Int. Cl. A61K 9/51; A61K 31/573; A61P 3/04; A61P 35/00

(52) U.S. Cl. CPC A61K 9/5115; A61P 35/00; A61P 3/04; A61K 31/573

(57) ABSTRACT

Provided herein are particles comprising compounds having a steroid core structure, or salts or esters thereof, and transition metal nanoparticles. Also provided herein are compositions comprising the particles, and methods of using the particles, for example in methods of treating liver disorders or for fat reduction.

### CROSS-REFERENCE TO RELATED APPLICATIONS

This application claims priority to U.S. Provisional Patent Application No. 62/874,784, filed on Jul. 16, 2019, the entire contents of which are fully incorporated herein by reference.

### BACKGROUND

Compounds having a steroid backbone, such as bile acids and corticosteroids, are useful in treating a wide variety of disorders. However, oral and subcutaneous administration of solubilized versions of these drugs may have limited efficacy and may impose unwanted side effects.

### SUMMARY

The present disclosure provides a composite particle comprising: (i) a compound having a steroid core structure, or a salt or ester thereof; and (ii) transition metal nanoparticles.

In some embodiments, the compound having a steroid core structure is selected from the group consisting of testosterone, exemestane, formestane, mesterolone, fluoxymesterone, methyltestosterone, oxandrolone, oxymetholone, mestranol, norethindrone, danazol, gestrinone, levonorgestrel, lynestrenol, norgestrel, desogestrel, etonogestrel, tibolone, ethynodiol, cyproterone, megestrol, abiraterone, dienogest, mifepristone, drospirenone, spironolactone, estradiol, polyestradiol, estramustine, estrone, estropipate, progesterone, dydrogesterone, hydroxyprogesterone, medroxyprogesterone, segesterone, norelgestromin, norgestimate, cortisol, cortisone, fluorometholone, difluprednate, fludrocortisone, fluocinolone, loteprednol, methylprednisolone, prednicarbate, prednisolone, prednisone, triamcinolone, alclometasone, betamethasone, clobetasol, clobetasone, clocortolone, desoximetasone, dexamethasone, diflorasone, difluocortolone, fluticasone, halometasone, mometasone, rimexolone, amcinonide, budesonide, ciclesonide, deflazacort, desonide, flunisolide, fluocinonide, halcinonide, cholesterol, estradiol, hydrocortisone, diflucortolone, boldenone, nandrolone, altrenogest, stanozolol, osaterone, estriol, aglepristone, trilostane, flumethasone, deoxycorticosterone, alfaxalone, desoxycorticosterone, and isoflupredone, or a salt or an ester thereof, or any combination thereof.

In some embodiments, the compound having a steroid core structure is a bile acid. In some embodiments, the bile acid is selected from the bile acid is selected from cholic acid, deoxycholic acid, chenodeoxycholic acid, lithocholic acid, glycocholic acid, taurocholic acid, glycodeoxycholic acid, taurodeoxycholic acid, glycochenodeoxycholic acid, taurochenodeoxycholic acid, glycolithocholic acid, taurolithocholic acid, ursodeoxycholic acid, glycoursodeoxycholic acid, tauroursodeoxycholic acid, and obeticholic acid. In some embodiments, the bile acid is selected from cholic acid, deoxycholic acid, ursodeoxycholic acid, and chenodeoxycholic acid. In some embodiments, the compound having a steroid core structure is a salt of a bile acid. In some embodiments, the salt of the bile acid is selected from sodium cholate, sodium deoxycholate, sodium ursodeoxycholate, and sodium chenodeoxycholate.

In some embodiments, the compound having a steroid core structure is a corticosteroid compound. In some embodiments, the corticosteroid compound is selected from hydrocortisone, dexamethasone, beclomethasone, ciclesonide, clobetasol, clobetasone, desonide, desoxymethasone, desoxycorticosterone, dichlorisone, diflorasone, diflucortolone, fluclarolone, fludrocortisone, flumethasone, fluocinolone, fluocinonide, flucortine, fluocortolone, fluprednidene, flurandrenolone, halcinonide, halometasone, methylprednisolone, triamcinolone, cortisone, cortodoxone, flucetonide, fluradrenalone, medrysone, alclometasone, amciafel, amcinafide, amcinonide, betamethasone, budesonide, chlorprednisone, clocortelone, clescinolone, difluprednate, flucloronide, flunisolide, fluoromethalone, fluperolone, fluprednisolone, hydrocortamate, meprednisone, mometasone, paramethasone, prednisolone, prednisone, prednicarbate, and tixocortol, or a salt or an ester thereof. In some embodiments, the corticosteroid compound is selected from methylprednisolone and hydrocortisone, or an ester thereof.

In some embodiments, the composite particle has a hexagonal prism shape. In some embodiments, the hexagonal prism has a diagonal length of 2.5 μm to 10 μm. In some embodiments, the hexagonal prism has a height of 2.5 μm to 6.5 μm.

In some embodiments, the composite particle has a rod shape. In some embodiments, the rod has a length of 2.5 μm to 100 μm. In some embodiments, the rod has a length of 10 μm to 50 μm.

In some embodiments, the composite particle has a hexagonal sheet shape. In some embodiments, the hexagonal sheet has a long side length of 10 μm to 50 μm, and a short side length of 5 μm to 20 μm.

In some embodiments, the composite particle has a spherical shape. In some embodiments, the sphere has a diameter of 1 μm to 10 μm.

In some embodiments, the transition metal nanoparticles are gold, silver, copper, platinum, palladium, nickel, or iron nanoparticles. In some embodiments, the transition metal nanoparticles are gold, silver, or copper nanoparticles. In some embodiments, the transition metal nanoparticles are gold nanoparticles. In some embodiments, the transition metal nanoparticles are silver nanoparticles. In some embodiments, the transition metal nanoparticles are copper nanoparticles.

In some embodiments, the particle consists essentially of the compound having a steroid core structure or a salt or ester thereof, and the transition metal nanoparticles. In some embodiments, the particle consists essentially of a bile acid or salt or ester thereof and gold nanoparticles.

The present disclosure also provides a composition comprising a plurality of composite particles described herein. In some embodiments, the composition further comprises a pharmaceutically acceptable carrier.

The present disclosure also provides a method of making a plurality of composite particles, comprising:
(a) providing a first solution a transition metal salt in water;
(b) adding a hydrophobic solvent to the solution and mixing to form a first emulsion;
(c) combining the first emulsion and a second solution, wherein the second solution comprises a compound having a steroid core structure or a salt or ester thereof, and mixing to form a second emulsion;
(d) combining the second emulsion and a third solution, wherein the third solution comprises the compound having a steroid core structure or a salt or ester thereof, to form a final mixture; and
(e) incubating the final mixture to form the plurality of composite particles.

In some embodiments, the second and third solutions comprise a salt of a bile acid. In some embodiments, the salt of the bile acid is selected from sodium cholate, sodium deoxycholate, sodium ursodeoxycholate, and sodium chenodeoxycholate. In some embodiments, the second and third solutions comprise a corticosteroid compound. In some embodiments, the corticosteroid compound is selected from methylprednisolone and hydrocortisone, or an ester thereof. In some embodiments, the first solution comprises a gold(III) salt, a silver(I) salt, a copper(II) salt, a nickel(II) salt, a palladium(II) salt, a platinum(II) salt, an iron(II) salt, or an iron(III) salt. In some embodiments, the first solution comprises HAuCl₄. In some embodiments, the compound having a steroid core structure is a bile acid or a salt or ester thereof, the metal salt is HAuCl₄, and the HAuCl₄ and the bile acid or salt or ester thereof are present in the final mixture in a mass ratio of at least 0.2. In some embodiments, the incubation step (e) comprises heating the final mixture at 40° C. to 100° C. for 10 minutes to 120 minutes. In some embodiments, the incubation step (e) comprises heating the final mixture at 45° C. for 15 minutes. In some embodiments, the method further comprises removing the solvent from the final mixture after the incubating step. In some embodiments, the hydrophobic solvent is ethyl acetate or dichloromethane. In some embodiments, the method further comprises separating the composite particles from the final mixture.

The present disclosure also provides a method of treating a liver disease or a peroxisomal disorder in a subject in need of treatment, comprising administering to the subject a therapeutically effective amount of a composition described herein (e.g., a composition comprising a plurality of composite particles described herein). In some embodiments, the liver disease is a bile acid synthesis disorder or primary biliary cholangitis. In some embodiments, the liver disease is a bile acid synthesis disorder due to a single enzyme defect. In some embodiments, the peroxisomal disorder is a Zellweger spectrum disorder.

The present disclosure also provides a method of non-surgical removal of a localized fat deposit in a subject, comprising contacting the deposit with an effective amount of a composition described herein (e.g., a composition comprising a plurality of composite particles described herein).

The present disclosure also provides a method of reducing a subcutaneous fat deposit in a subject in need thereof, comprising administering locally to the subcutaneous fat deposit in the subject an effective amount of a composition described herein (e.g., a composition comprising a plurality of composite particles described herein).

The present disclosure also provides a method of treating cancer in a subject in need thereof, comprising administering to the subject an effective amount of a composition described herein (e.g., a composition comprising a plurality of composite particles described herein). In some embodiments, the cancer is colorectal cancer or gastric cancer.

The present disclosure also provides a method of reducing the proliferation of cancer cells, comprising contacting the cells with an effective amount of a composition described herein (e.g., a composition comprising a plurality of composite particles described herein). In some embodiments, the cancer cells are colorectal cancer cells or gastric cancer cells.

The present disclosure also provides a method of treating a disorder selected from the group consisting of endocrine disorders, rheumatic disorders, collagen diseases, dermatologic diseases, allergic states, ophthalmic diseases, respiratory diseases, hematologic disorders, neoplastic diseases, gastrointestinal diseases, nervous system disorders, inflammatory disorders, and renal diseases, comprising administering to the subject a therapeutically effective amount of a composition described herein (e.g., a composition comprising a plurality of composite particles described herein).

The present disclosure also provides uses of the particles and compositions described herein (e.g., use for removal of a localized fat deposit, use for reducing a subcutaneous fat deposit in a subject, use in the treatment of cancer such as colorectal cancer, use in reducing the proliferation of cancer cells, use in the treatment of disorders selected from endocrine disorders, rheumatic disorders, collagen diseases, dermatologic diseases, allergic states, ophthalmic diseases, respiratory diseases, hematologic disorders, neoplastic diseases, gastrointestinal diseases, nervous system disorders, inflammatory disorders, and renal diseases, etc.).

### BRIEF DESCRIPTION OF THE DRAWINGS

FIG. 1 shows an exemplary synthesis of cholate-based composite particles via a double emulsion solvent evaporation technique.

FIGS. 2A-2D show: (A) a scanning electron microscopy (SEM) image of cholate-based composite particles fabricated with the modified emulsion solvent evaporation technique as described in Example 1; (B) a brightfield microscopy image of the emulsion droplets after the reaction proceeded for 0 minutes, with an inset showing an image of the reaction vial; (C) a brightfield microscopy image of the emulsion droplets after the reaction proceeded for 10 minutes, with an inset showing an image of the reaction vial; and (D) a brightfield microscopy image of the emulsion droplets after the reaction proceeded for 15 minutes, with an inset showing an image of the reaction vial.

FIGS. 3A-3D show: (A) an SEM image of cholate-based product before any separation; (B) energy-dispersive spectroscopy (EDS) analysis of the cholate-based hexagon particles; (C) EDS analysis of gold nanoparticles; and (D) X-ray photoelectron spectroscopy (XPS) analysis of the dried cholate hexagon particles mounted on indium foil.

FIGS. 4A-4B shows a fluorescence microscopy image of: (A) rhodamine-loaded cholate-based particles, and (B) rhodamine-loaded deoxycholate-based particles, both prepared as described in Example 1; the scale bars are 100 μm.

FIG. 5 shows a brightfield microscopy image of cholate-based particles prepared using dichloromethane as a solvent; the scale bar is 20 μm.

FIGS. 6A-6B show microscopy images of deoxycholate-based composite particles prepared as described in Example 1: (A) a brightfield microscopy image of deoxycholate-based composite particles; and (B) SEM image of deoxycholate-based composite particles (scale bar 1 μm)

FIG. 7 shows an SEM image of ursodeoxycholate-based composite particles prepared as described in Example 1 (scale bar 10 μm).

FIGS. 8A-8B show data for chenodeoxycholate composite particles prepared as described in Example 1: (A) SEM image of chenodeoxycholate composite particles; (B) EDS analysis of the chenodeoxycholate composite particles.

FIGS. 9A-9D show: (A) HPLC analysis of the degradation products from cholate-based particles and a standard sodium cholate solution in a 50:50 mixture of acetonitrile and water; ¹H NMR spectra of (B) a 3% standard sodium cholate solution and the degradation product of cholate-based particles, and (C) a 3% standard sodium deoxycholate solution and the degradation product of deoxycholate-based particles in D₂O (with the intensities of the peaks for the degraded cholate/deoxycholate-based particles increased using MestReNova software for ease of comparison); and (D) a release profile of rhodamine from deoxycholate composite particles fabricated in the presence of different amount of HAuCl₄, where the concentration of the particles in PBS was set at 10 mg/mL.

FIGS. 10A-10B show: (A) SEM images of the cholate-based particles during a degradation assay as described in Example 3; and (B) quantified amounts of released cholate after incubation of cholate-based particles at 37° C. for different time points.

FIGS. 11A-11E show SEM images of cholate-based particles of different sizes as described in Example 4, being fabricated in the presence of: (A) 0.75% w/v, (B) 2% w/v, (C) 3% w/v, (D) 0.5% w/v, and (E) 10% w/v sodium cholate in the outer water phase. The scale bars are 1 μm for FIGS. 11A-11D and 100 μm for FIG. 11E.

FIGS. 12A-12B show: (A) a brightfield microscopy image of particles after the heating step when 6-carboxyfluorescein was added to the inner water phase, as described in Example 4; (B) an SEM image of the elongated bipyramidal hexagon particles fabricated in the presence of 6-carboxyfluorescein, as described in Example 4.

FIGS. 13A-13D show cell viability data for HUVECs after incubation with: (A) different concentrations of sodium deoxycholate solution in culture media for 1 hour; (B) different numbers of the composite cholate-based composite particles per well for 1 hour; (C) 10⁶ composite particles per well incubated for different time-points; and (D) 0.1% of the deoxycholate salt and 10⁶ cholate particles in culture media and 5% BSA solutions for 3 hr.

FIGS. 14A-14D show cell viability data for primary subcutaneous adipocytes after being incubated with: (A) different concentrations of the deoxycholate and cholate salts in RPMI media; (B) different concentrations of the cholate-based and deoxycholate-based composite particles for different time points; (C) 10⁵ cholate or deoxycholate particles in RPMI media or 5% BSA; and (D) either the cholate or deoxycholate particles, or the supernatant from a reaction in which either cholate or deoxycholate particles had been preincubated in media at 37° C. for 3 hours, as described in Example 6.

FIGS. 15A-15D show: (A) the visual appearance of 0.5 gr beef adipose tissue after incubation with PBS, 1% sodium deoxycholate, and different concentrations of the cholate particles; (B) turbidity measurements after incubation with PBS, 1% sodium deoxycholate, and different concentrations of the cholate particles; (C) measurements of the free fatty acids from 0.5 gr beef adipose tissue after being incubated with PBS, 1% sodium cholate, 1% sodium deoxycholate, 10⁷ cholate-based, and 10⁷ deoxycholate-based composite particles; and (D) the visual appearance of chicken breast after incubation with PBS, 1% sodium deoxycholate, and different concentrations of the cholate particles.

FIGS. 16A-16H show the visual appearance of obese animals after receiving 100 μL of (A) pure saline, (B) sodium deoxycholate, (C) one dosage, and (D) two dosages of deoxycholate microparticles in saline at different times post-injection. 2.5 mg of the salt or the particles were injected into the right inguinal fat pad of animals in each trial. The purple arrows show the formation of an ulcer at the injection site in animals that had received the sodium deoxycholate injection. Post-euthanasia appearance of the inguinal fat pads of animals 2-weeks after receiving (E) pure saline, (F) sodium deoxycholate, (G) one dosage, and (H) two dosages of deoxycholate particles. Purple arrows show lipolysis sites in the right fat pad, and red arrows show the remains of particles at the injection site.

FIG. 17 shows data for the weight of obese mice at different time points after receiving sodium deoxycholate salt or deoxycholate particles.

FIGS. 18A-18D show histology H&E sections of the adipose tissue for animals that had received (A) pure saline, (B) sodium deoxycholate, (C) one dosage, and (D) two dosages of deoxycholate microparticles. 8-10 week old female B6.Cg-Lep^{ob}/J animals were used for all the trials.

FIGS. 19A-19C show histology sections of the left inguinal fat pad of obese animals that had received lipolytic treatment in their right fat pad.

FIG. 20 shows brightfield microscopy images of HCT-116 colon cancer cells after being incubated with various concentrations of cholate composite microparticles.

FIGS. 21A-21B show data for the viability of HCT-116 colon cancer cells after being incubated with: (A) various concentrations of cholate-based composite microparticles for 1 hour; and (B) with 5×10⁶ particles/well of a 24-well plate for various time points in media.

FIGS. 22A-22B show data for silver-templated cholate composite particles prepared as described in Example 10: (A) SEM image of the particles; (B) EDS analysis of the particles.

FIGS. 23A-23B show data for copper-templated cholate composite particles prepared as described in Example 11: (A) SEM image of the particles; (B) EDS analysis of the particles.

FIGS. 24A-24B show data for gold-templated methylprednisolone composite particles prepared as described in Example 12: (A) SEM image of the particles; (B) EDS analysis of the particles.

FIGS. 25A-25B show data for gold-templated hydrocortisone composite particles prepared as described in Example 13: (A) SEM image of the particles; (B) EDS analysis of the particles.

FIG. 26 shows images of a gold-templated sodium cholate particle under a brightfield lens and a polarized lens.

### DETAILED DESCRIPTION

The present disclosure relates to particles that may be used for controlled release of compounds having a steroid core structure, or their salts. The particles may be used for a variety of medical and dermatological treatments, such as treatments of liver disorders and for non-surgical removal of localized fat deposits. The particles can also be used for the treatment of cancer or in a method of reducing proliferation of cancer cells.

### Composite Particles

In this regard, in one aspect, the disclosure provides a composite particle comprising: (i) a compound having a steroid core structure, or a salt or ester thereof; and (ii) transition metal nanoparticles.

The composite particles include a compound having a steroid core structure, as shown below, with the conventional numbering on the perhydrocyclopenta[a]phenanthrene core:

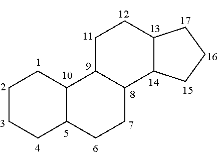 [SMILES: C1CCC2C(C1)CCC1C3CCCC3CCC21]

The steroid core structure can be fully saturated as shown above, or can include one or more double bonds. The core structure can include one or more alkyl functional groups; for example, steroid compounds contain methyl groups at the C10 and C13 positions, and often contain an alkyl group (or a functionalized alkyl group) at C17. The core structure can also include one or more hydroxy or oxo groups; for example, steroids and sterols have an oxo or hydroxy group at C3.

In some embodiments, the compound having a steroid core structure is selected from the group consisting of testosterone (e.g., testosterone enanthate, testosterone cypionate, or testosterone undecanoate), exemestane, formestane, mesterolone, fluoxymesterone, methyltestosterone, oxandrolone, oxymetholone, mestranol, norethindrone, danazol, gestrinone, levonorgestrel, lynestrenol, norgestrel, desogestrel, etonogestrel, tibolone, ethynodiol (e.g., ethynodiol diacetate), cyproterone, megestrol (e.g., megestrol acetate), abiraterone (e.g., abiraterone acetate), dienogest, mifepristone, drospirenone, spironolactone, estradiol, polyestradiol phosphate, estramustine (e.g., estramustine phosphate), estrone, estropipate, progesterone, dydrogesterone, hydroxyprogesterone (e.g., hydroxyprogesterone caproate), medroxyprogesterone (e.g., medroxyprogesterone acetate), segesterone (e.g., segesterone acetate), norelgestromin, norgestimate, cortisol, cortisone, fluorometholone, difluprednate, fludrocortisone (e.g., fludrocortisone acetate), fluocinolone (e.g., fluocinolone acetonide), loteprednol (e.g., loteprednol etabonate), methylprednisolone (e.g., methylprednisolone acetate or methylprednisolone succinate), prednicarbate, prednisolone (e.g., prednisolone sodium phosphate or prednisolone acetate), prednisone, triamcinolone (e.g., triamcinolone acetonide or triamcinolone hexacetonide), alclometasone (e.g., alclometasone diproprionate), betamethasone (e.g., betamethasone sodium phosphate, betamethasone benzoate, betamethasone dipropionate, betamethasone valerate, or betamethasone acetate), clobetasol (e.g., clobetasol propionate), clobetasone (e.g., clobetasone butyrate), clocortolone (e.g., clocortolone pivalate), desoximetasone, dexamethasone (e.g., dexamethasone phosphate or dexamethasone sodium phosphate), diflorasone (e.g., diflorasone diacetate), difluocortolone, fluticasone (fluticasone propionate or fluticasone furoate), halometasone, mometasone (e.g., mometasone furoate), rimexolone, amcinonide, budesonide, ciclesonide, deflazacort, desonide, flunisolide, fluocinonide, halcinonide, cholesterol, estradiol valerate, hydrocortisone (e.g., hydrocortisone acetate, hydrocortisone buteprate, hydrocortisone butyrate, hydrocortisone succinate, or hydrocortisone valerate), diflucortolone (e.g., diflucortolone valerate), boldenone (e.g., boldenone undecylenate), nandrolone, altrenogest, stanozolol, osaterone (e.g., osaterone acetate), estriol, aglepristone, trilostane, flumethasone, deoxycorticosterone, alfaxalone, desoxycorticosterone (e.g., desoxycorticosterone pivalate), and isoflupredone (e.g., isoflupredone acetate), or any combination thereof.

In some embodiments, the composite particles comprise the compound having a steroid core structure or salt or ester thereof in an amount of about 70 wt % to about 99 wt %, or about 80 wt % to about 95 wt %. For example, in some embodiments the composite particles comprise the compound having a steroid core structure or salt or ester thereof in an amount of about 70 wt %, 71 wt %, 72 wt %, 73 wt %, 74 wt %, 75 wt %, 76 wt %, 77 wt %, 78 wt %, 79 wt %, 80 wt %, 81 wt %, 82 wt %, 83 wt %, 84 wt %, 85 wt %, 86 wt %, 87 wt %, 88 wt %, 89 wt %, 90 wt %, 91 wt %, 92 wt %, 93 wt %, 94 wt %, 95 wt %, 96 wt %, 97 wt %, 98 wt %, or 99 wt %, or any range therebetween.

In some embodiments, the compound having a steroid core structure is a bile acid, or a salt or ester thereof. In such embodiments, the disclosure provides a composite particle comprising: (i) a bile acid, or a salt or ester thereof; and (ii) transition metal nanoparticles. In some embodiments, the disclosure provides a composite particle comprising a bile acid, or a salt or ester thereof, and gold nanoparticles.

Bile acids are steroid acids primarily found in bile, including both primary bile acids, which are synthesized by the liver, and secondary bile acids, which are synthesized from primary bile acids by bacteria in the colon. Bile acids and their salts help to solubilize lipids in the small intestine and regulate several hepatic, biliary, and intestinal functions; they have been proposed as therapeutic agents for treatment of different conditions including bile acid synthesis disorders and peroxisomal disorders, primary biliary cholangitis, primary sclerosing cholangitis, cardiometabolic diseases, gallstones and bile duct stones, non-alcoholic fatty liver disease, type-2 diabetes, human immunodeficiency virus type 1 (HIV-1), acute pancreatitis, and cancer.

Orally administered formulations of certain bile acids have been approved by the United States Food and Drug Administration (FDA) for treatment of different bile synthesis disorders and liver dysfunctions. For example, cholic acid capsules are approved for treatment of bile acid synthesis disorders and peroxisomal disorders, and both ursodeoxycholic acid tablets and obeticholic acid tablets have been approved for treatment of primary biliary cholangitis. However, oral administration of these drugs may limit their bioavailability and can impose cytotoxicity risks through their membrane disruptive properties. Additionally, injectable deoxycholic acid is approved by the U.S. FDA for destruction of fat cells to reduce moderate-to-severe fat below the chin. However, numerous injections are required for effective treatment. Formulation of bile acids in the composite particles disclosed herein provide controlled or targeted delivery of bile acids to increase specificity while lowering side effects.

Bile acids may be conjugated with taurine or glycine. Exemplary bile acids include cholic acid, deoxycholic acid, chenodeoxycholic acid, lithocholic acid, glycocholic acid, taurocholic acid, glycodeoxycholic acid, taurodeoxycholic acid, glycochenodeoxycholic acid, taurochenodeoxycholic acid, glycolithocholic acid, taurolithocholic acid, ursodeoxycholic acid, glycoursodeoxycholic acid, and tauroursodeoxycholic acid. Bile acids also include semi-synthetic bile acids, such as obeticholic acid. In some embodiments, the bile acid is selected from cholic acid, deoxycholic acid, ursodeoxycholic acid, and chenodeoxycholic acid. In some embodiments, the bile acid is cholic acid. In some embodiments, the bile acid is deoxycholic acid. In some embodiments, the bile acid is ursodeoxycholic acid. In some embodiments, the bile acid is chenodeoxycholic acid. In some embodiments, the composite particles comprise a combination of two or more bile acids or salts thereof.

The bile acid may be in the form of a salt. In some embodiments, the bile acid salt is a sodium or potassium salt. In some embodiments, the bile acid salt is a sodium salt. Accordingly, in some embodiments, the particle comprises a bile acid salt selected from sodium cholate, sodium deoxycholate, sodium ursodeoxycholate, and sodium chenodeoxycholate. In some embodiments, the bile acid salt is sodium cholate. In some embodiments, the bile acid salt is sodium deoxycholate. In some embodiments, the bile acid salt is sodium ursodeoxycholate. In some embodiments, the bile acid salt is sodium chenodeoxycholate.

In some embodiments, the composite particles comprise the bile acid or salt or ester thereof in an amount of about 70 wt % to about 99 wt %, or about 80 wt % to about 95 wt %. For example, in some embodiments the composite particles comprise the bile acid or salt or ester thereof in an amount of about 70 wt %, 71 wt %, 72 wt %, 73 wt %, 74 wt %, 75 wt %, 76 wt %, 77 wt %, 78 wt %, 79 wt %, 80 wt %, 81 wt %, 82 wt %, 83 wt %, 84 wt %, 85 wt %, 86 wt %, 87 wt %, 88 wt %, 89 wt %, 90 wt %, 91 wt %, 92 wt %, 93 wt %, 94 wt %, 95 wt %, 96 wt %, 97 wt %, 98 wt %, or 99 wt %, or any range therebetween.

In some embodiments, the compound having a steroid core structure is a corticosteroid, or a salt or ester thereof. In such embodiments, the disclosure provides a composite particle comprising: (i) a corticosteroid, or a salt or ester thereof; and (ii) transition metal nanoparticles. In some embodiments, the disclosure provides a composite particle comprising a corticosteroid, or a salt or ester thereof, and gold nanoparticles.

Corticosteroids are a class of steroid hormones produced in the adrenal cortex of vertebrates, as well as synthetic analogs of such hormones. The two main categories of corticosteroids are glucocorticoids and mineralocorticoids. These compounds are involved in a wide range of physiological processes, including stress response, immune response, and regulation of inflammation, carbohydrate metabolism, protein catabolism, blood electrolyte levels, and behavior.

Corticosteroids are generally grouped into four classes: Group A (hydrocortisone type), including hydrocortisone (e.g., hydrocortisone acetate, hydrocortisone aceponate, hydrocortisone buteprate, hydrocortisone butyrate, hydrocortisone cypionate, or hydrocortisone succinate), cortisone (e.g., cortisone acetate), tixocortol (e.g., tixocortol pivalate), prednisolone, methylprednisolone (e.g., methylprednisolone succinate), and prednisone; Group B (acetonides and related compounds), including amcinonide, budesonide, desonide, fluocinolone (e.g., fluocinolone acetonide), fluocinonide, halcinonide, and triamcinolone (e.g., triamcinolone acetonide); Group C (betamethasone type), including beclomethasone, betamethasone, dexamethasone, fluocortolone, halometasone, and mometasone; and Group D (esters), including Group D₁ halogenated esters (alclometasone dipropionate, betamethasone dipropionate, betamethasone valerate, clobetasol propionate, clobetasone butyrate, fluprednidene acetate, and mometasone furoate), and Group D₂ labile prodrug esters (ciclesonide, cortisone acetate, hydrocortisone aceponate, hydrocortisone acetate, hydrocortisone buteprate, hydrocortisone butyrate, hydrocortisone valerate, prednicarbate, and tixocortol pivalate). In some embodiments, the corticosteroid is selected from hydrocortisone (e.g., hydrocortisone acetate, hydrocortisone aceponate, hydrocortisone buteprate, hydrocortisone butyrate, hydrocortisone cypionate, or hydrocortisone succinate), dexamethasone (e.g., dexamethasone phosphate), beclometasone (e.g., beclometasone dipropionate), ciclesonide, clobetasol (e.g., clobetasol propionate or clobetasol valerate), clobetasone (e.g., clobetasone butyrate), desonide, desoxymethasone, desoxycorticosterone (e.g., desoxycorticosterone acetate), dichlorisone, diflorasone (e.g., diflorasone diacetate), diflucortolone (e.g., diflucortolone valerate), fluadrenolone, fluclarolone (e.g., fluclarolone acetonide), fludrocortisone (e.g., fludrocortisone acetate), flumethasone (e.g., flumethasone pivalate), fluocinolone (e.g., fluocinolone acetonide), fluocinonide, flucortine (e.g., flucortine butylester), fluocortolone, fluprednidene (e.g., fluprednideneacetate), flurandrenolone, halcinonide, halometasone, methylprednisolone (e.g., methylprednisolone succinate), triamcinolone (e.g., triamcinolone acetonide), cortisone (e.g., cortisone acetate), cortodoxone, flucetonide, fluradrenalone (e.g., fluradrenalone acetonide), medrysone, alclometasone (e.g., alclometasone dipropionate), amciafel, amcinafide, amcinonide, betamethasone (e.g., betamethasone dipropionate or betamethasone valerate), budesonide, chlorprednisone (e.g., chlorprednisone acetate), clocortelone, clescinolone, difluprednate, flucloronide, flunisolide, fluoromethalone, fluperolone, fluprednisolone, hydrocortamate, meprednisone, mometasone (e.g., mometasone furoate), paramethasone, prednisolone, prednisone, prednicarbate, and tixocortol (e.g., tixocortol pivalate). In some embodiments, the corticosteroid is selected from methylprednisolone (e.g., methylprednisolone succinate) and hydrocortisone (e.g., hydrocortisone succinate).

In some embodiments, the composite particles comprise the corticosteroid or salt or ester thereof in an amount of about 70 wt % to about 99 wt %, or about 80 wt % to about 95 wt %. For example, in some embodiments the composite particles comprise the bile acid or salt or ester thereof in an amount of about 70 wt %, 71 wt %, 72 wt %, 73 wt %, 74 wt %, 75 wt %, 76 wt %, 77 wt %, 78 wt %, 79 wt %, 80 wt %, 81 wt %, 82 wt %, 83 wt %, 84 wt %, 85 wt %, 86 wt %, 87 wt %, 88 wt %, 89 wt %, 90 wt %, 91 wt %, 92 wt %, 93 wt %, 94 wt %, 95 wt %, 96 wt %, 97 wt %, 98 wt %, or 99 wt %, or any range therebetween.

The compound having a steroid core structure can be in the form of a salt. For example, the compound having a steroid core structure can have one or more acidic moieties (e.g., carboxylates), which can form a salt with a suitable cation, such as an alkali metal cation (e.g., sodium, lithium, potassium), an ammonium cation (e.g., NR₄⁺, where each R is independently selected from hydrogen and an alkyl group), or the like. In some embodiments, the salt is a sodium salt. The compound having a steroid core structure also includes esters of steroid compounds. In such ester compounds, one or more hydroxy groups can be functionalized with an acyl group to form an ester. Exemplary ester groups include acetate, adamantoate, benzoate, buteprate, butyrate, caproate, cypionate, enanthate, etabonate, furoate, hexanoate, linoleate, palmitate, pivalate, propionate, tebutate, succinate, undecanoate, undecylenate, valerate, and the like. The compound can also be in the form of a cyclic ketal, such as a cyclic acetal. Compounds with a steroid core structure often have two adjacent hydroxy groups, which can form a cyclic acetal to form an acetonide (e.g., with acetone, particularly at the C16 and C17 positions).

In addition to the compound having a steroid core structure (or salt or ester thereof), the particles further comprise transition metal nanoparticles. In some embodiments, the particles comprise gold, silver, copper, platinum, palladium, nickel, or iron nanoparticles. In some embodiments, the particles comprise gold, silver, or copper nanoparticles. In some embodiments, the particles comprise gold nanoparticles. In some embodiments, the transition metal nanoparticles have an average particle diameter of about 1 nm to about 500 nm, or about 10 nm to about 300 nm. For example, in some embodiments the transition metal nanoparticles have an average particle diameter of about 1 nm, about 2 nm, about 3 nm, about 4 nm, about 5 nm, about 6 nm, about 7 nm, about 8 m, about 9 nm, about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 110 nm, about 120 nm, about 130 nm, about 140 nm, about 150 nm, about 160 nm, about 170 nm, about 180 nm, about 190 nm, about 200 nm, about 250 nm, about 300 nm, about 350 nm, about 400 nm, about 450 nm, or about 500 nm.

The transition metal nanoparticles may be present in the particles in an amount of about 1 wt % to about 30 wt %, or about 5 wt % to about 20 wt %. For example, in some embodiments the composite particles comprise the transition metal nanoparticles in an amount of about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 19 wt %, about 20 wt %, about 21 wt %, about 22 wt %, about 23 wt %, about 24 wt %, about 25 wt %, about 26 wt %, about 27 wt %, about 28 wt %, about 29 wt %, or about 30 wt %, or any range there between.

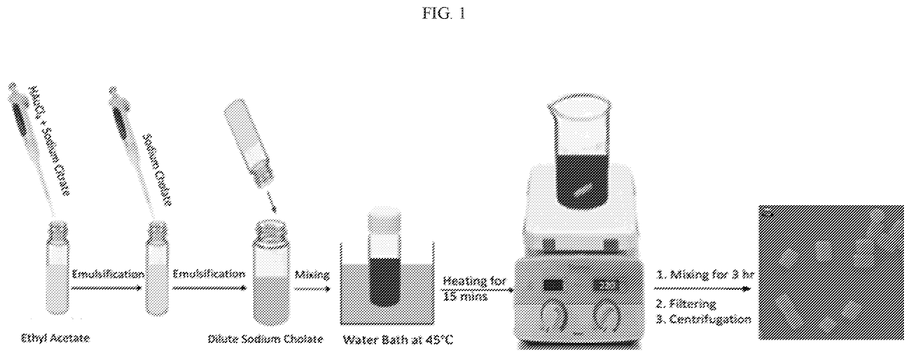

In some embodiments, the nanoparticles are produced during the particle fabrication process, which involves a modified double emulsion solvent evaporation method with in situ reduction of metal ions (e.g., Au(III), Ag(I), or Cu(II) ions) at the oil-water interface. An exemplary process is illustrated in FIG. 1, in which the inner water phase of the system is doped with Au(III) ions (e.g., from HAuCl₄) and sodium citrate. According to the Turkevich method (see, e.g., Kimling et al. J. Phys. Chem. B, 2006, 110, 15700-15707, which is incorporated herein by reference), reduction of the metal ion precursor in the inner water phase of the emulsion, initiated by heating the emulsion, results in formation of the metal nanoparticles and further enables formation of the composite particles. The particles self-assemble at the oil-water interface, and the final product of after complete evaporation of the organic solvent will be the composite particles of the compound having a steroid core structure, which further include the metal nanoparticles. The composite particles can be separated from free metal nanoparticles via a number of methods, such as low-speed or high-speed centrifugation and recovering the pellet, or filtration.

Accordingly, in one aspect, the disclosure provides a method of making a plurality of particles, the method comprising:
(a) providing a first solution of a transition metal salt in water;
(b) adding a hydrophobic solvent to the solution and mixing to form a first emulsion;
(c) combining the first emulsion and a second solution, wherein the second solution comprises a compound having a steroid core structure, or a salt or ester thereof, and mixing to form a second emulsion;
(d) combining the second emulsion and a third solution, wherein the third solution comprises the compound having a steroid core structure, or a salt or ester thereof, to form a final mixture; and
(e) incubating the final mixture to form the plurality of particles.

In some embodiments, the first solution comprises a gold(III) salt, a silver(I) salt, a copper(II) salt, a platinum(II) salt, a palladium(II) salt, a nickel(II) salt, an iron(II) salt, or an iron(III) salt. In some embodiments, the first solution comprises a gold(III) salt, a silver(I) salt, or a copper(II) salt. In some embodiments, the first solution comprises HAuCl₄. In some embodiments, the first solution comprises a mixture of HAuCl₄ and sodium citrate. In some embodiments, the first solution comprises a silver(I) salt. In some embodiments, the first solution comprises silver nitrate. In some embodiments, the first solution comprises a copper(II) salt. In some embodiments, the first solution comprises copper(II) chloride.

In some embodiments, the disclosure provides a method of making a plurality of particles, the method comprising:
(a) providing a first solution of sodium citrate and HAuCl₄ in water;
(b) adding a hydrophobic solvent to the solution and mixing to form a first emulsion;
(c) combining the first emulsion and a second solution, wherein the second solution comprises a bile acid or a salt or ester thereof, and mixing to form a second emulsion;
(d) combining the second emulsion and a third solution, wherein the third solution comprises the bile acid or a salt or ester thereof, to form a final mixture; and
(e) incubating the final mixture to form the plurality of particles.

In some embodiments, the second and third solutions comprise a salt of a bile acid selected from sodium cholate, sodium deoxycholate, sodium ursodeoxycholate, and sodium chenodeoxycholate. In some embodiments, the HAuCl₄ and the bile acid or salt or ester thereof are present in the final mixture in a mass ratio of at least 0.2.

In some embodiments, the incubation step (e) comprises of no heating. In such embodiments, step (e) comprises incubating the sample at room temperature for about 2 hours to about 10 hours, or about 4 hours to about 5 hours, e.g., about 2 hours, about 3 hours, about 4 hours, about 5 hours, about 6 hours, about 7 hours, about 8 hours, about 9 hours, or about 10 hours.

In some embodiments, the incubation step (e) comprises heating the final mixture at a temperature of about 40° C. to about 100° C., for example, about 40° C., about 45° C., about 50° C., about 55° C., about 60° C., about 65° C., about 70° C., about 75° C., about 80° C., about 85° C., about 90° C., about 95° C., or about 100° C. In some embodiments, step (e) comprises heating the final mixture at a temperature of about 40° C. to about 50° C., for example, about 41° C., about 42° C., about 43° C., about 44° C., about 45° C., about 46° C., about 47° C., about 48° C., about 49° C., or about 50° C.

Also in step (e), the mixture may be heated for about 10 minutes to about 120 minutes, for example, about 10, 15, 20, 25, 30, 35, 40, 45, 50, 55, 60, 65, 70, 75, 80, 85, 90, 95, 100, 105, 110, 115, or 120 minutes. In some embodiments, step (e) comprises heating the final mixture for about 10 minutes to about 20 minutes, for example, about 10, 11, 12, 13, 14, 15, 16, 17, 18, 19, or 20 minutes. In some embodiments, step (e) comprises heating the final mixture at a temperature of about 45° C. for about 15 minutes. In some embodiments, step (e) comprises heating the final mixture at a temperature of about 80° C. for about 60 minutes. In some embodiments, step (e) comprises heating the final mixture at a temperature of about 80° C. for about 90 minutes. In some embodiments, step (e) comprises no heating; in such embodiments, step (e) comprises incubating the sample at room temperature (i.e., at 25° C.) for about 2-10 hours (e.g., about 4 hours).

The heating step may be conducted, for example, by immersing a reaction vessel containing the final mixture in a water bath that is heated to the indicated temperature.

The hydrophobic solvent used in step (b) may be any suitable hydrophobic solvent that is compatible with the other components of the mixture. In some embodiments, the hydrophobic solvent is ethyl acetate or dichloromethane. In some embodiments, the hydrophobic solvent is ethyl acetate. In some embodiments, the hydrophobic solvent is dichloromethane.

In some embodiments, the method further comprises removing the solvent from the final mixture after the incubation step. The solvent can be removed using a variety of methods, such as evaporation at ambient temperature and pressure, or evaporation with heating, or evaporation under reduced pressure. In some embodiments, the solvent is removed by evaporation at ambient temperature and pressure.

The method may further comprise an additional step of separating the composite particles from the mixture. The separating step can include filtering using a filter having an appropriate pore size. The separating step may alternatively or additionally include centrifugation. For example, low-speed centrifugation (e.g., at 100-1000 rpm, such as 100, 200, 300, 400, 500, 600, 700, 800, 900, or 1000 rpm) may effectively separate free metal nanoparticles from the composite particles. The composite particles may be recovered in the pellet from the centrifugation, while the free metal nanoparticles remain in the supernatant.

Specific methods of making composite particles disclosed herein are described in the Examples. Additional methods of making transition metal nanoparticles are known in the art. See, for example: Chen et al. J. Phys. Chem. C 2010, 114, 50, 21976-21981 (for palladium nanoparticles); Jeyaraj et al. Nanomaterials-Basel 2019, 9(12): 1719 (for platinum nanoparticles); Huber, Small 2005, 1(5): 482-501 (for iron nanoparticles); and Hou et al. Appl. Surf Sci. 2005, 241(1-2), 218-222 (for nickel nanoparticles).

In some embodiments, provided herein are particles produced by any of the above described methods.

In some embodiments, the composite particles consist essentially of a compound having a steroid core structure, or the salt or ester thereof, and transition metal nanoparticles. In some embodiments, the particle consists essentially of a bile acid or salt or ester thereof and gold, silver, copper, platinum, palladium, nickel, or iron nanoparticles. In some embodiments, the composite particles consist essentially of a bile acid or a salt or ester thereof, and gold, silver, or copper nanoparticles. In some embodiments, the composite particles consist essentially of a bile acid or a salt or ester thereof, and gold nanoparticles. In some embodiments, the composite particles consist essentially of a corticosteroid, or a salt or ester thereof, and transition metal nanoparticles. In some embodiments, the particle consists essentially of a corticosteroid, or a salt or ester thereof, and gold, silver, copper, platinum, palladium, nickel, or iron nanoparticles. In some embodiments, the composite particles consist essentially of a corticosteroid, or a salt or ester thereof, and gold, silver, or copper nanoparticles. In some embodiments, the composite particles consist essentially of a corticosteroid, or a salt or ester thereof, and gold nanoparticles. In such embodiments, the composite particles do not include, or are substantially free of, other components such as small molecules, polymers, and the like. In some embodiments, the composite particles do not include a polymer. For example, the composite particles do not include polymers such as polyesters, including poly(lactic-co-glycolic acid) (PLGA), or anionic polymers such as polysaccharides (e.g., dextran sulfate, heparin, heparin sulfate, chondroitin sulfate, hyaluronic acid, or alginic acid), nucleic acid polymers, and the like. For example, in the composite particles of the disclosure, the compound having the steroid core structure (e.g., the bile acid or the corticosteroid) is not conjugated to a polymer. In some embodiments, the composite particles do not include phosphatidylcholine.

In other embodiments, the particles may further comprise other components. For example, in some embodiments, the particles further include a targeting ligand. Targeting ligands are well-known to those skilled in the art; exemplary targeting ligands are described by Srinivasarao et al. “Ligand-Targeted Drug Delivery,” Chem. Rev. 2017, 117(19), 12133-12164, which is incorporated herein by reference. Exemplary targeting ligands include antibodies to molecules that are expressed on cell surfaces; for example, certain lipoma cells are known to overexpress CD34, and an anti-CD34 antibody could be used as a targeting ligand for those cells.

In other embodiments, the particles further include an additional therapeutic agent. Any suitable therapeutic agent can be used. Exemplary therapeutic agents include those described in Harrison's Principles of Internal Medicine, 20th Edition, Eds. J. L. Jameson et al., McGraw-Hill Education (2018); Physicians' Desk Reference, 71st Edition, PDR Network (2017); and Goodman & Gilman's The Pharmacological Basis of Therapeutics, 13^{th} Edition, Eds. L. L. Brunton et al., 2017; United States Pharmacopeia—The National Formulary, USP 42-NF 37, 2019; the contents of each of which are incorporated herein by reference.

The composite particles can be characterized by a wide variety of techniques. For example, particles can be imaged using scanning electron microscopy (SEM) to determine their size and shape. The elemental composition can be confirmed using elemental analysis, for example using energy-dispersive spectroscopy (EDS) and/or X-ray photoelectron spectroscopy (XPS). The presence of the bile acid can further be confirmed using techniques such as high-performance liquid chromatography (HPLC), Fourier-transform infrared (FTIR) spectroscopy, and nuclear magnetic resonance (NMR) spectroscopy.

In some embodiments, the composite particles have a hexagonal prism shape. Such particles can be characterized by their diagonal length, i.e. the length between two opposite vertices of the hexagon, as well as their height. In some embodiments, the composite particles have a hexagonal prism shape with an average diagonal length of about 2.5 μm to about 10 μm, or about 3.0 μm to about 9.0 μm (e.g., about 2.5, 3.0, 3.5, 4.0, 4.5, 5.0, 5.5, 6.0, 6.5, 7.0, 7.5, 8.0, 8.5, 9.0, 9.5, or 10 μm, or any range therebetween). In some embodiments, the composite particles have a hexagonal prism shape with an average height of about 2.5 μm to about 6.5 μm (e.g., about 2.5, 3.0, 3.5, 4.0, 4.5, 5.0, 5.5, 6.0, or 6.5 μm, or any range therebetween).

In some embodiments, the composite particles have a rod shape. In some embodiments, the composite particles have a rod shape with an average length of about 2.5 μm to about 100 μm, or about 10 μm to about 50 μm (e.g., about 2.5, 5.0, 10, 15, 20, 25, 30, 35, 40, 45, 50, 55, 60, 65, 70, 75, 80, 85, 90, 95, or 100 μm, or any range therebetween).

In some embodiments, the composite particles have a hexagonal sheet shape. Such particles can be characterized by the length of the long and short sides of the sheet. In some embodiments, the composite particles have a hexagonal sheet shape with an average long side length of about 10 μm to about 50 μm (e.g., about 10, 15, 20, 25, 30, 35, 40, 45, or 50 μm, or any range therebetween), and a short side length of 5 μm to 20 μm (e.g., about 5, 7.5, 10, 12.5, 15, 17.5, or 20 μm, or any range therebetween).

In some embodiments, the composite particles have a spherical shape. In some embodiments, the sphere has a diameter of 1 μm to 10 μm (e.g., about 1 μm, about 2 μm, about 3 μm, about 4 μm, about 5 μm, about 6 μm, about 7 μm, about 8 μm, about 9 μm, or about 10 μm, or any range therebetween).

### Compositions

In another aspect, the disclosure provides a composition comprising a plurality of the composite particles described herein. In some embodiments, the compositions further comprise a pharmaceutically acceptable carrier.

As used herein, the term “pharmaceutically acceptable carrier” refers to a pharmaceutically-acceptable material, composition or vehicle for administration of an active agent described herein. Pharmaceutically acceptable carriers include any and all solvents, dispersion media, coatings, antibacterial and antifungal agents, isotonic and absorption delaying agents, and the like which are compatible with the activity of the bile acid or salt or ester thereof and are physiologically acceptable to the subject. Some examples of materials that can serve as pharmaceutically-acceptable carriers include: (i) sugars, such as lactose, glucose and sucrose; (ii) starches, such as corn starch and potato starch; (iii) cellulose, and its derivatives, such as sodium carboxymethyl cellulose, methylcellulose, ethyl cellulose, microcrystalline cellulose and cellulose acetate; (iv) powdered tragacanth; (v) malt; (vi) gelatin; (vii) lubricating agents, such as magnesium stearate, sodium lauryl sulfate and talc; (viii) excipients, such as cocoa butter and suppository waxes; (ix) oils, such as peanut oil, cottonseed oil, safflower oil, sesame oil, olive oil, com oil and soybean oil; (x) glycols, such as propylene glycol; (xi) polyols, such as glycerin, sorbitol, mannitol and polyethylene glycol (PEG); (xii) esters, such as ethyl oleate and ethyl laurate; (xiii) agar; (xiv) buffering agents, such as magnesium hydroxide and aluminum hydroxide; (xv) alginic acid; (xvi) pyrogen-free water; (xvii) isotonic saline; (xviii) Ringer's solution; (xix) ethyl alcohol; (xx) pH buffered solutions; (xxi) polyesters, polycarbonates and/or polyanhydrides; (xxii) bulking agents, such as polypeptides and amino acids (xxiii) serum component, such as serum albumin, HDL and LDL; (xxiv) C2-C12 alcohols, such as ethanol; and (xxv) other non-toxic compatible substances employed in pharmaceutical formulations. Wetting agents, coloring agents, release agents, coating agents, sweetening agents, flavoring agents, perfuming agents, preservative and antioxidants can also be present in the formulation.

For formulations described herein to be administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while com starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

Pharmaceutically acceptable carriers can vary in a formulation described herein, depending on the administration route. The formulations described herein can be delivered via any administration mode known to a skilled practitioner. For example, the formulations described herein can be delivered in a systemic manner, via administration routes such as, but not limited to, oral and parenteral, including intravenous, intramuscular, intraperitoneal, intradermal, and subcutaneous. In some embodiments, the formulations described herein are in a form that is suitable for injection, particularly subcutaneous injection. In other embodiments, the formulations described herein are formulated for oral administration, such as a tablet or a capsule.

When administering parenterally, a formulation described herein can be generally formulated in a unit dosage injectable form (solution, suspension, emulsion). The formulations suitable for injection include sterile aqueous solutions or dispersions. The carrier can be a solvent or dispersing medium containing, for example, water, buffers (e.g., phosphate buffered saline), polyol (for example, glycerol, propylene glycol, liquid polyethylene glycol, and the like), salts (e.g., sodium chloride), or suitable mixtures thereof. In some embodiments, the pharmaceutical carrier can be a saline solution. In some embodiments, the pharmaceutical carrier can be a buffered solution (e.g., PBS). Where necessary, the composition may also include a solubilizing agent and a local anesthetic such as lignocaine to ease pain at the site of the injection.

The formulations can also contain auxiliary substances such as wetting or emulsifying agents, pH buffering agents, gelling or viscosity enhancing additives, preservatives, colors, and the like, depending upon the route of administration and the preparation desired. Standard texts, such as “REMINGTON'S PHARMACEUTICAL SCIENCE,” 17th edition, 1985, incorporated herein by reference, may be consulted to prepare suitable preparations, without undue experimentation. With respect to formulations described herein, however, any vehicle, diluent, or additive used should have to be biocompatible with the active agents described herein. Those skilled in the art will recognize that the components of the formulations should be selected to be biocompatible with respect to the active agent. This will present no problem to those skilled in chemical and pharmaceutical principles, or problems can be readily avoided by reference to standard texts or by simple experiments (not involving undue experimentation).

For in vivo administration, the formulations described herein can be administered with a delivery device, e.g., a syringe. Accordingly, an additional aspect described herein provides for delivery devices comprising at least one chamber with an outlet, wherein the at least one chamber comprises a pre-determined amount of any formulation described herein and the outlet provides an exit for the formulation enclosed inside the chamber. In some embodiments, a delivery device described herein can further comprise an actuator to control release of the formulation through the outlet. Such delivery device can be any device to facilitate the administration of any formulation described herein to a subject, e.g., a syringe, a dry powder injector, a nasal spray, a nebulizer, or an implant such as a microchip, e.g., for sustained-release or controlled release of any formulation described herein.

In some embodiments, the compositions do not include phosphatidylcholine.

### Methods of Use

The composite particles can be used in a variety of methods, such as a methods of treating a disorder in a subject.

As used herein, the term “subject” includes human and non-human animals. Exemplary human subjects include a human patient having a disorder, e.g., a disorder described herein, or a normal subject. The term “non-human animals” includes all vertebrates, e.g., non-mammals (such as chickens, amphibians, reptiles) and mammals, such as non-human primates, domesticated and/or agriculturally useful animals, e.g., horse, sheep, dog, cat, cow, pig, etc.

As used herein, the term “treat” or “treating” a subject having a disorder refers to subjecting the subject to a regimen, e.g., the administration of a particle or a composition described herein, such that at least one symptom of the disorder is cured, healed, alleviated, relieved, altered, remedied, ameliorated, or improved. Treating includes administering an amount effective to alleviate, relieve, alter, remedy, ameliorate, improve or affect the disorder or the symptoms of the disorder. The treatment may inhibit deterioration or worsening of a symptom of a disorder.

Bile acids and their salts have been proposed as therapeutic agents for treatment of different conditions including bile acid synthesis disorders and peroxisomal disorders (see, e.g., Klouwer et al. Orphanet J. Rare Dis., 2015, 10, 151; W. T. Elliot. Internal Medicine Alert, 2015, 37), primary biliary cholangitis (also known as primary biliary cirrhosis; see, e.g., Hirschfield et al. Gut, 2018, 67, 1568), primary sclerosing cholangitis (see, e.g., Mikov et al. Eur. J. Drug Metab. Pharmacokinet., 2006, 31, 237), cardiometabolic diseases (see, e.g., Ikemoto et al. Am. J. Physiol. Metab., 1997, 273, E37), gallstones and bile duct stones (see, e.g., Fini et al. J. Pharm. Sci. 85(9), 971); Lansford et al. Gut, 1974, 15, 48), non-alcoholic fatty liver disease (see, e.g., Quintero et al. J. Physiol. Biochem., 2014, 70, 667; Gabbi et al. Dig. Liver Dis., 2012, 44, 1018), type-2 diabetes (see, e.g., Gabbi 2012), human immunodeficiency virus type 1 (HIV-1) (see, e.g., Mikov 2006), acute pancreatitis (see, e.g., Mikov 2006), cancer (see, e.g., Goossens et al. Pharmacol. Ther. 203, 107396 (2019); Zeng et al. Nutr. Cancer 62, 85-92 (2009); Pardi et al. Gastroenterology 124, 889-893 (2003); Milovic et al. Eur. J. Clin. Invest. 32, 29-34 (2002); Schlottmann et al. Cancer Res. 60, 4270-4276 (2000); Šarenac et al. Front. Pharmacol. 10:484 (2019), doi: 10.3389/fphar.2019.00484), and removal of undesired fat (Yagima Odo et al. Dermatologic Surg. 33, 178-189 (2007)). Bile acids and salts thereof are also useful for the non-surgical removal of a localized fat deposit in a subject. For example, deoxycholic acid injections have been approved by the U.S. FDA for improving the appearance of moderate to severe convexity or fullness associated with submental fat in adults. However, many injections are needed, with a single treatment including up to 50 injections, and up to six single treatments may be needed for effective treatment. Accordingly, it is contemplated that the composite particles disclosed herein provide controlled release of the bile acid or salt or ester thereof, which reduces the number of injections required for effective treatment.

Corticosteroids are used to treat a variety of conditions, including endocrine disorders (e.g., primary or secondary adrenocortical insufficiency, congenital adrenal hyperplasia, nonsuppurative thyroiditis, or hypercalcemia associated with cancer), rheumatic disorders (e.g., rheumatoid arthritis (including juvenile rheumatoid arthritis), ankylosing spondylitis, acute and subacute bursitis, synovitis of osteoarthritis, acute nonspecific tenosynovitis, post-traumatic osteoarthritis, psoriatic arthritis, epicondylitis, or acute gouty arthritis), collagen diseases (e.g., during an exacerbation or as maintenance therapy in systemic lupus erythematosus, systemic dermatomyositis (polymyositis), or acute rheumatic carditis), dermatologic diseases (e.g., bullous dermatitis herpetiformis, severe erythema multiforme (Stevens-Johnson syndrome), severe seborrheic dermatitis, exfoliative dermatitis, mycosis fungoides, pemphigus, or severe psoriasis), allergic states (e.g., seasonal or perennial allergic rhinitis, drug hypersensitivity reactions, serum sickness, contact dermatitis, bronchial asthma, or atopic dermatitis), ophthalmic diseases (e.g., severe acute and chronic allergic and inflammatory processes involving the eye and its adnexa, such as allergic corneal marginal ulcers, herpes zoster ophthalmicus, anterior segment inflammation, diffuse posterior uveitis and choroiditis, sympathetic ophthalmia, keratitis, optic neuritis, allergic conjunctivitis, chorioretinitis, uveitis and ocular inflammatory conditions unresponsive to topical steroids, or iritis and iridocyclitis), respiratory diseases (e.g., symptomatic sarcoidosis, berylliosis, Loeffler's syndrome, fulminating or disseminated pulmonary tuberculosis, aspiration pneumonitis, chronic obstructive pulmonary disease (COPD), allergic bronchopulmonary aspergillosis, asthma, hypersensitivity pneumonitis, idiopathic bronchiolitis obliterans with organizing pneumonia, idiopathic eosinophilic pneumonias, idiopathic pulmonary fibrosis, or Pneumocystis carinii pneumonia), hematologic disorders (e.g., idiopathic thrombocytopenic purpura, secondary thrombocytopenia, acquired (autoimmune) hemolytic anemia, erythroblastopenia (RBC anemia), congenital (erythroid) hypoplastic anemia, Diamond-Blackfan anemia, or pure red cell aplasia), neoplastic diseases (e.g., leukemias and lymphomas in adults, or acute leukemia of childhood), gastrointestinal diseases (e.g., during acute episodes of ulcerative colitis, regional enteritis, or Crohn's disease), nervous system disorders (e.g., acute exacerbations of multiple sclerosis), inflammatory disorders, renal diseases (e.g., to induce a diuresis or remission of proteinuria in nephrotic syndrome of the idiopathic type or that is due to lupus erythematosus), and other conditions (e.g., tuberculous meningitis with subarachnoid block or impending block when used concurrently with appropriate antituberculous chemotherapy, trichinosis with neurological or myocardial involvement). Corticosteroids are often used for treatment of severe allergies or skin problems, asthma, and arthritis.

Other compounds having steroid core structures are also used in treating a wide variety of disorders. For example, listed below are compounds having steroid core structures, and disorders they are used to treat in humans. In some embodiments, the composite particles described herein comprise the indicated compounds, and the disclosure provides a method of using such particles to treat the indicated disorders and/or for the following purposes:

Testosterone (Testosterone, testosterone enanthate, and testosterone cypionate forms): Breast cancer, metastatic; delayed puberty; Hypogonadism, hypogonadotropic (congenital or acquired); Hypogonadism, primary (congenital or acquired); Hormone therapy for transgender males (female-to-male);

Testosterone undecanoate: Breast cancer, metastatic; Delayed puberty; Hypogonadism, hypogonadotropic (congenital or acquired); Hypogonadism, primary (congenital or acquired); Hormone therapy for transgender males (female-to-male);

Exemestane: Breast cancer; First-line adjuvant treatment of estrogen receptor-positive early breast cancer in postmenopausal women; Risk reduction for invasive breast cancer in postmenopausal women;

Formestane: Treatment of advanced breast cancer in postmenopausal women;

Mesterolone: Androgen deficiency; hypogonadism; infertility; delayed puberty;

Fluoxymesterone: Breast cancer, metastatic (females); Delayed puberty (males); Hypogonadism, primary or hypogonadotrophic (males);

Methyltestosterone: Breast cancer, metastatic (females); Delayed puberty (males); Hypogonadism, primary or hypogonadotropic (males);

Oxandrolone: Weight gain (adjunctive therapy); Burns, severe (adjunctive therapy);

Oxymetholone: Anemia; Fanconi anemia;

Mestranol (administered with norethindrone): Abnormal uterine bleeding; Dysmenorrhea; Menstrual bleeding (menorrhagia); Pain associated with endometriosis; Polycystic ovary syndrome (PCOS) in women with menstrual irregularities and hirsutism/acne;

Norethindrone: Abnormal uterine bleeding; Amenorrhea, secondary; Contraception; Endometriosis;

Danazol: Endometriosis; Hereditary angioedema (HAE), prophylaxis; Cyclic breast pain (mastalgia) associated with benign breast disorders; Immune thrombocytopenia, refractory;

Gestrinone: Endometriosis;

Levonorgestrel: Contraception; Heavy menstrual bleeding; Endometrial hyperplasia;

Lynestrenol: Prevention of pregnancy; treatment of polymenorrhea, menorrhagia, metrorrhagia, primary and secondary amenorrhea or oligomenorrhea; treatment of benign breast disease; treatment of endometrial carcinoma; adjunct to estrogen therapy in peri- and post-menopausal women to prevent endometrial hyperplasia; treatment of endometriosis; suppression of ovulation, ovulation pain or menstruation, or dysmenorrhea; to postpone the onset of menstruation;

Norgestrel (administered with ethinyl estradiol): Contraception; Abnormal uterine bleeding; Dysmenorrhea; Hirsutism; Menstrual bleeding (menorrhagia); Pain associated with endometriosis; Polycystic ovary syndrome (PCOS) in women with menstrual irregularities and hirsutism/acne;

Desogestrel: Contraception; Abnormal uterine bleeding; Dysmenorrhea; Hirsutism; Menstrual bleeding (menorrhagia); Polycystic ovary syndrome (PCOS) in women with menstrual irregularities and hirsutism/acne;

Etonogestrel: Contraception;

Tibolone: Treatment of symptoms associated with menopause; prevention of postmenopausal osteoporosis in high-risk women with contraindications or an intolerance to first-line therapy;

Ethynodiol diacetate (administered with ethinyl estradiol): Contraception; Abnormal uterine bleeding; Dysmenorrhea; Hirsutism; Menstrual bleeding (menorrhagia); Pain associated with endometriosis; Polycystic ovary syndrome (PCOS) in women with menstrual irregularities and hirsutism/acne;

Cyproterone: Prostate cancer; Hormone therapy for transgender females (male-to-female); Paraphilia;

Megestrol acetate: Anorexia or cachexia; Breast cancer; Endometrial cancer; Treatment of cancer-related cachexia;

Abiraterone acetate: Prostate cancer, metastatic;

Dienogest: Endometriosis;

Mifepristone: To control hyperglycemia occurring secondary to hypercortisolism in adult patients with endogenous Cushing syndrome who have type 2 diabetes mellitus or glucose intolerance and who failed surgery or who are not surgical candidates; Medical termination of intrauterine pregnancy through 70 days gestation, in combination with misoprostol; Early pregnancy loss;

Drospirenone: Contraception;

Spironolactone: Ascites due to cirrhosis; Heart failure with reduced ejection fraction; Hypertension; Primary hyperaldosteronism; Acne vulgaris, females; Heart failure with preserved ejection fraction; Heart failure with reduced ejection fraction; Hirsutism, females; Hormone therapy for transgender females, male-to-female; Post myocardial infarction, complicated by reduced ejection fraction;

Estradiol: Breast cancer, metastatic; Hypoestrogenism (female); Osteoporosis prevention (female); Prostate cancer, advanced; Vasomotor symptoms associated with menopause; Vulvar and vaginal atrophy associated with menopause; Functional hypothalamic amenorrhea with low bone density (young adult females); Hormone therapy for transgender females (male-to-female);

Polyestradiol phosphate: Palliative treatment of advanced, inoperable carcinoma of the prostate;

Megestrol: Anorexia or cachexia; Breast cancer; Endometrial cancer; Treatment of cancer-related cachexia;

Estramustine: Prostate cancer (metastatic castration-resistant);

Estramustine phosphate: Prostate Cancer;

Estrone: Vulvar and vaginal atrophy;

Estropipate: Hypoestrogenism, female; Osteoporosis prevention; Vasomotor symptoms due to menopause; Vulvar and vaginal atrophy due to menopause;

Progesterone: Prevention of endometrial hyperplasia in nonhysterectomized, postmenopausal women who are receiving conjugated estrogens; treatment of secondary amenorrhea; Treatment of amenorrhea or abnormal uterine bleeding due to hormonal imbalance in the absence of organic pathology, such as submucous fibroids or uterine cancer; Part of assisted reproductive technology (ART) for infertile women with progesterone deficiency; To support embryo implantation and early pregnancy by supplementation of corpus luteal function as part of ART for infertile women; Reduce the risk of recurrent spontaneous preterm birth;

Dydrogesterone: Treatment of various conditions caused by progesterone deficiencies;

Hydroxyprogesterone caproate: To reduce the risk of preterm birth in women with a singleton pregnancy who have a history of singleton spontaneous preterm birth; Treatment of advanced (stage III or IV) uterine adenocarcinoma; management of amenorrhea (primary and secondary) and abnormal uterine bleeding due to hormonal imbalance in the absence of organic pathology (e.g., submucous fibroids or uterine cancer); as a test for endogenous estrogen production; production of secretory endometrium and desquamation;

Medroxyprogesterone acetate: Abnormal uterine bleeding; Amenorrhea, secondary; Contraception; Endometrial hyperplasia prevention; Endometrial carcinoma; Endometriosis; Abnormal uterine bleeding, acute; Endometrial hyperplasia; Hot flashes; Paraphilia/hypersexuality;

Segesterone acetate (administered with ethinyl estradiol): Contraceptive;

Norelgestromin (administered with ethinyl estradiol): Contraception; Polycystic ovary syndrome (PCOS) in women with menstrual irregularities and hirsutism/acne;

Norgestimate (administered with estradiol): Osteoporosis prevention; Vasomotor symptoms associated with menopause; Vulvar and vaginal atrophy associated with menopause;

Norgestimate (administered with ethinyl estradiol): Acne vulgaris; Contraception; Abnormal uterine bleeding; Dysmenorrhea; Hirsutism; Menstrual bleeding (menorrhagia); Polycystic ovary syndrome (PCOS) in women with menstrual irregularities and hirsutism/acne;

Cortisol: Allergic states: Control of severe or incapacitating allergic conditions intractable to adequate trials of conventional treatment in drug hypersensitivity reactions, perennial or seasonal allergic rhinitis, serum sickness, transfusion reactions, or acute noninfectious laryngeal edema (epinephrine is the drug of first choice); Dermatologic diseases: Atopic dermatitis; bullous dermatitis herpetiformis; contact dermatitis; exfoliative dermatitis; exfoliative erythroderma; pemphigus; severe erythema multiforme (Stevens-Johnson syndrome); severe psoriasis; severe seborrheic dermatitis; mycosis fungoides; Edematous states: To induce diuresis or remission of proteinuria in the nephrotic syndrome, without uremia, of the idiopathic type or that due to lupus erythematosus; Endocrine disorders: Acute adrenocortical insufficiency; congenital adrenal hyperplasia; hypercalcemia associated with cancer; nonsuppurative thyroiditis; primary or secondary adrenocortical insufficiency; preoperatively and in the event of serious trauma or illness, in patients with known adrenal insufficiency or when adrenocortical reserve is doubtful; shock unresponsive to conventional therapy if adrenocortical insufficiency exists or is suspected; GI diseases: To tide the patient over a critical period of the disease in ulcerative colitis and regional enteritis; Hematologic disorders: Acquired (autoimmune) hemolytic anemia; congenital (erythroid) hypoplastic anemia (Diamond Blackfan anemia); erythroblastopenia (RBC anemia); immune thrombocytopenia (formerly known as idiopathic thrombocytopenic purpura) in adults; pure red cell aplasia; select cases of secondary thrombocytopenia; Neoplastic diseases: Palliative management of leukemias and lymphomas (adults); acute leukemia of childhood; Nervous system: Cerebral edema associated with primary or metastatic brain tumor, or craniotomy; Ophthalmic diseases: Severe acute and chronic allergic and inflammatory processes involving the eye, such as allergic conjunctivitis; allergic corneal marginal ulcers; anterior segment inflammation; chorioretinitis; diffuse posterior uveitis and choroiditis; herpes zoster ophthalmicus; iritis and iridocyclitis; keratitis; optic neuritis; sympathetic ophthalmia; other ocular inflammatory conditions unresponsive to topical corticosteroids; Respiratory diseases: Aspiration pneumonitis; bronchial asthma; berylliosis; fulminating or disseminated pulmonary tuberculosis when used concurrently with appropriate antituberculous chemotherapy; idiopathic eosinophilic pneumonias; Loeffler syndrome (not manageable by other means); symptomatic sarcoidosis; Rheumatic disorders: As adjunctive therapy for short-term administration in acute and subacute bursitis, acute gouty arthritis, acute nonspecific tenosynovitis, ankylosing spondylitis, epicondylitis, posttraumatic osteoarthritis, psoriatic arthritis, rheumatoid arthritis, including juvenile rheumatoid arthritis, synovitis of osteoarthritis; during an exacerbation or as maintenance therapy in acute rheumatic carditis, dermatomyositis (polymyositis), temporal arteritis, and systemic lupus erythematosus; Miscellaneous: Trichinosis with neurologic or myocardial involvement; tuberculous meningitis with subarachnoid block or impending block when used concurrently with appropriate antituberculous chemotherapy; In-hospital cardiac arrest; Septic shock; Thyroid storm;

Cortisone (Cortisone and cortisone acetate forms): Allergic states: Control of severe or incapacitating allergic conditions intractable to adequate trials of conventional treatment of atopic dermatitis, bronchial asthma, contact dermatitis, drug hypersensitivity reactions, seasonal or perennial allergic rhinitis, and serum sickness; Dermatologic diseases: Bullous dermatitis herpetiformis, exfoliative dermatitis, mycosis fungoides, pemphigus, severe erythema multiforme (Stevens-Johnson syndrome), severe psoriasis, severe seborrheic dermatitis; Endocrine disorders: Congenital adrenal hyperplasia, hypercalcemia associated with cancer, nonsuppurative thyroiditis, primary or secondary adrenocortical; Gastrointestinal diseases: To tide the patient over a critical period of the disease in regional enteritis and ulcerative colitis; Hematologic disorders: Acquired (autoimmune) hemolytic anemia, congenital (erythroid) hypoplastic anemia, erythroblastopenia (red blood cell [RBC] anemia), immune thrombocytopenia (formerly known as idiopathic thrombocytopenic purpura) in adults, secondary thrombocytopenia in adults; Neoplastic diseases: Palliative management of leukemias and lymphomas in adults; acute leukemia of childhood; Ophthalmic diseases: Severe acute and chronic allergic and inflammatory processes involving the eye and its adnexa (e.g., allergic conjunctivitis, allergic corneal marginal ulcers, anterior segment inflammation, chorioretinitis, diffuse posterior uveitis and choroiditis, keratitis, herpes zoster ophthalmicus, iritis and iridocyclitis, optic neuritis, sympathetic ophthalmia); Renal diseases: To induce diuresis or remission of proteinuria in nephrotic syndrome, without uremia, of the idiopathic type or that is caused by lupus erythematosus; Respiratory diseases: Aspiration pneumonitis, berylliosis, fulminating or disseminated pulmonary tuberculosis when used concurrently with appropriate antituberculosis chemotherapy, Loeffler syndrome not manageable by other means, symptomatic sarcoidosis; Rheumatic disorders: Adjunctive therapy for short-term administration (to tide the patient over an acute episode or exacerbation) in acute and subacute bursitis; acute gouty arthritis; acute nonspecific tenosynovitis; ankylosing spondylitis; epicondylitis; posttraumatic osteoarthritis; psoriatic arthritis; rheumatoid arthritis (RA), including juvenile RA (select cases may require low-dose maintenance therapy); and synovitis of osteoarthritis. During an exacerbation or as maintenance therapy in select cases of acute rheumatic carditis, systemic dermatomyositis (polymyositis), and systemic lupus erythematosus; Miscellaneous: Tuberculous meningitis with subarachnoid block or impending block when used concurrently with appropriate antituberculous chemotherapy; trichinosis with neurologic or myocardial involvement;

Fluorometholone: Ocular inflammation: Treatment of steroid-responsive inflammation of the palpebral and bulbar conjunctiva, cornea, and anterior segment of the eye;

Difluprednate: Inflammation/pain: Treatment of inflammation and pain following ocular surgery; Uveitis: Treatment of endogenous anterior uveitis;

Fludrocortisone (Fludrocortisone acetate form): Adrenal insufficiency, primary; Congenital adrenal hyperplasia, classic; Idiopathic orthostatic hypotension; Septic shock;

Fluocinolone (fluocinolone acetonide form): Body oil: Treatment of moderate to severe atopic dermatitis in pediatric patients ≥3 months; treatment of atopic dermatitis in adults; Cream, ointment, topical solution: Relief of inflammatory and pruritic manifestations of corticosteroid-responsive dermatoses; Scalp oil: Treatment of psoriasis of the scalp in adults; Shampoo: Treatment of seborrheic dermatitis of the scalp; Relief of chronic eczematous external otitis; Diabetic macular edema; Uveitis;

Loteprednol (Loteprednol etabonate form): Ophthalmic inflammatory conditions (0.5% suspension): Treatment of ocular, anterior segment inflammation that is expected to be responsive to topical corticosteroid therapy; Postoperative inflammation/pain (0.38% gel; 0.5% suspension/ointment/gel; 1% suspension): Treatment of postoperative inflammation and pain following ocular surgery; Seasonal allergic conjunctivitis (0.2% suspension): Temporary relief of signs and symptoms of seasonal allergic conjunctivitis;

Methylprednisolone (methylprednisolone acetate and methylprednisolone succinate forms): Oral, IM (acetate or succinate), and IV (succinate only) administration: Anti-inflammatory or immunosuppressant agent in the treatment of a variety of diseases, including those of hematologic (e.g., immune thrombocytopenia, warm autoimmune hemolytic anemia), allergic, gastrointestinal (e.g., Crohn disease, ulcerative colitis), inflammatory, neoplastic, neurologic (e.g., multiple sclerosis), rheumatic (e.g., antineutrophil cytoplasmic antibody-associated vasculitis, dermatomyositis/polymyositis, giant-cell arteritis, gout [acute flare], giant cell arteritis, mixed cryoglobulinemia syndrome, polyarteritis nodosa, rheumatoid arthritis, systemic lupus erythematosus), and/or autoimmune origin; Intra-articular or soft tissue administration (acetate only): Gout (acute flare), acute and subacute bursitis, acute nonspecific tenosynovitis, epicondylitis, rheumatoid arthritis, and/or synovitis of osteoarthritis; Intralesional administration (acetate only): Alopecia areata; discoid lupus erythematosus; keloids; localized hypertrophic, infiltrated, inflammatory lesions of granuloma annulare, lichen planus, lichen simplex chronicus (neurodermatitis), and psoriatic plaques; and necrobiosis lipoidica diabeticorum. May be useful in cystic tumor of an aponeurosis or tendon (ganglia); Acute respiratory distress syndrome, moderate to severe; Cardiac transplant: Antibody-mediated rejection; Chronic obstructive pulmonary disease; Deceased organ donor management; Graft-vs-host disease, acute; In-hospital cardiac arrest; Nausea and vomiting of pregnancy, severe/refractory; Pneumocystis pneumonia, adjunctive therapy for moderate to severe disease; Prostate cancer, metastatic, castration-resistant;

Prednicarbate: Dermatoses;

Prednisolone (Prednisolone sodium phosphate and prednisolone acetate forms): Corneal injury: Treatment of acute chemical injury of the cornea; Ophthalmic inflammatory conditions: Treatment of ocular, anterior segment inflammation that is expected to be responsive to topical corticosteroid therapy; Allergic states: Control of severe or incapacitating allergic conditions intractable to adequate trials of conventional treatment in asthma, atopic dermatitis, drug hypersensitivity reactions, seasonal or perennial allergic rhinitis, and serum sickness; Dermatologic diseases: Bullous dermatitis herpetiformis; contact dermatitis; exfoliative erythroderma; exfoliative dermatitis; mycosis fungoides; pemphigus; severe erythema multiforme (Stevens-Johnson syndrome); severe psoriasis; severe seborrheic dermatitis; Endocrine disorders: Congenital adrenal hyperplasia; hypercalcemia associated with cancer; nonsuppurative thyroiditis; primary or secondary adrenocortical insufficiency; GI diseases: During acute episodes of Crohn disease or ulcerative colitis; Hematologic disorders: Acquired (autoimmune) hemolytic anemia; congenital (erythroid) hypoplastic anemia (Diamond-Blackfan anemia); erythroblastopenia (RBC anemia); immune thrombocytopenia (formerly known as idiopathic thrombocytopenic purpura), pure red cell aplasia; secondary thrombocytopenia; Neoplastic diseases: Treatment of acute leukemia and aggressive lymphomas; Nervous system: Acute exacerbations of multiple sclerosis; cerebral edema associated with primary or metastatic brain tumor, craniotomy, or head injury; Ophthalmic diseases: Allergic conjunctivitis; allergic corneal marginal ulcers; anterior segment inflammation; chorioretinitis; diffuse posterior uveitis and choroiditis; herpes zoster ophthalmicus; iritis and iridocyclitises; keratitis; optic neuritis; sympathetic ophthalmia; uveitis and other ocular inflammatory conditions unresponsive to topical corticosteroids; Renal disorders: To induce diuresis or remission of proteinuria in nephrotic syndrome, without uremia, of the idiopathic type or that due to lupus erythematosus; Respiratory diseases: Acute exacerbations of chronic obstructive pulmonary disease (COPD); allergic bronchopulmonary aspergillosis; aspiration pneumonitis; asthma; berylliosis; fulminating or disseminated pulmonary tuberculosis when used concurrently with appropriate antituberculous chemotherapy; hypersensitivity pneumonitis; idiopathic bronchiolitis obliterans with organizing pneumonia; idiopathic eosinophilic pneumonias; idiopathic pulmonary fibrosis; Loeffler syndrome (not manageable by other means); Pneumocystis carinii pneumonia (PCP) associated with hypoxemia occurring in an HIV-positive individual who is also under treatment with appropriate anti-PCP antibiotics; symptomatic sarcoidosis; Rheumatic disorders: As adjunctive therapy for short-term administration in acute and subacute bursitis, acute gout flares, acute nonspecific tenosynovitis, ankylosing spondylitis, epicondylitis, polymyalgia rheumatica/temporal arteritis, posttraumatic osteoarthritis, psoriatic arthritis, relapsing polychondritis, rheumatoid arthritis (including juvenile rheumatoid arthritis), synovitis of osteoarthritis, acute rheumatic carditis, systemic lupus erythematosus, dermatomyositis/polymyositis, Sjogren syndrome, and certain cases of vasculitis; Miscellaneous: Acute or chronic solid organ rejection; trichinosis with neurologic or myocardial involvement; tuberculous meningitis with subarachnoid block or impending block, tuberculosis with enlarged mediastinal lymph nodes causing respiratory difficulty, tuberculosis with pleural or pericardial effusion (use appropriate antituberculous chemotherapy concurrently when treating any tuberculosis complications); Alcoholic hepatitis (severe); Asthma exacerbation; Bell palsy; Chronic obstructive pulmonary disease (COPD) (acute exacerbation);

Prednisone: Anti-inflammatory or immunosuppressant agent in the treatment of a variety of diseases, including allergic, hematologic (e.g., immune thrombocytopenia, warm autoimmune hemolytic anemia), dermatologic, GI, inflammatory, ophthalmic, neoplastic, rheumatic (e.g., acute gout flare, vasculitis, dermatomyositis, mixed cryoglobulinemia syndrome, polyarteritis nodosa, polymyositis, polymyalgia rheumatica, rheumatoid arthritis, systemic lupus erythematosus), autoimmune, nervous system (e.g., acute exacerbations of multiple sclerosis), renal, respiratory (e.g., asthma), and endocrine (e.g., primary or secondary adrenocorticoid deficiency); solid organ rejection (acute/chronic); Bell palsy, new onset; Chronic spontaneous urticaria, acute exacerbation; Duchenne muscular dystrophy; Giant cell arteritis, treatment; Graft-versus-host disease, acute, treatment; Hepatitis, autoimmune; Minimal change disease, treatment; Multiple myeloma (previously untreated; transplant-ineligible); Myasthenia gravis, crisis; Pericarditis, acute; Pneumocystis pneumonia, adjunctive therapy for moderate to severe disease; Prostate cancer, metastatic; Takayasu arteritis; Thyroiditis, subacute; Tuberculosis, pulmonary;

Triamcinolone (Triamcinolone acetonide form): Allergic rhinitis; Upper respiratory allergies; Acute bacterial rhinosinusitis, adjunct to antibiotics (empiric treatment); Chronic rhinosinusitis; Alopecia areata; discoid lupus erythematosus; keloids; localized hypertrophic, infiltrated, inflammatory lesions of granuloma annulare, lichen planus, lichen simplex chronicus (neurodermatitis), and psoriatic plaques; necrobiosis lipoidica diabeticorum; cystic tumors of an aponeurosis or tendon (ganglia); Allergic states: Control of severe or incapacitating allergic conditions intractable to adequate trials of conventional treatment in asthma, drug hypersensitivity reactions, perennial or seasonal allergic rhinitis, serum sickness, or transfusion reactions; Dermatologic diseases: Atopic dermatitis, bullous dermatitis herpetiformis, contact dermatitis, exfoliative erythroderma, mycosis fungoides, pemphigus, or severe erythema multiforme (Stevens-Johnson syndrome), vulvar dermatitis, psoriasis, seborrheic dermatitis; Endocrine disorders: Primary or secondary adrenocortical insufficiency, congenital adrenal hyperplasia, hypercalcemia associated with cancer, or nonsuppurative thyroiditis; GI diseases: To tide the patient over a critical period of disease in Crohn disease or ulcerative colitis; Hematologic disorders: Acquired (autoimmune) hemolytic anemia, Diamond-Blackfan anemia, pure red cell aplasia, select cases of secondary thrombocytopenia; Neoplastic diseases: Palliative management of leukemias and lymphomas; Nervous system: Acute exacerbations of multiple sclerosis; cerebral edema associated with primary or metastatic brain tumor or craniotomy; Ophthalmic diseases: Sympathetic ophthalmia, temporal arteritis, uveitis, and ocular inflammatory conditions unresponsive to topical corticosteroids; Renal diseases: To induce diuresis or remission of proteinuria in idiopathic nephrotic syndrome or that is caused by lupus erythematosus; Respiratory diseases: Berylliosis, fulminating or disseminated pulmonary tuberculosis when used concurrently with appropriate antituberculous chemotherapy, idiopathic eosinophilic pneumonias, symptomatic sarcoidosis; Rheumatic disorders: As adjunctive therapy for short-term administration in acute gout flares; acute rheumatic carditis; ankylosing spondylitis; psoriatic arthritis; RA, including juvenile RA; treatment of dermatomyositis, polymyositis, and systemic lupus erythematosus; Miscellaneous: Trichinosis with neurologic or myocardial involvement; tuberculous meningitis with subarachnoid block or impending block when used with appropriate antituberculous chemotherapy; Aphthous stomatitis;

Alclometasone (Alclometasone diproprionate form): Steroid-responsive dermatosis;

Betamethasone (Betamethasone, betamethasone sodium phosphate, betamethasone benzoate, betamethasone dipropionate, betamethasone valerate, and betamethasone acetate forms): Allergic states: Control of severe or incapacitating allergic conditions intractable to adequate trials of conventional treatment in asthma, atopic dermatitis, contact dermatitis, drug hypersensitivity reactions, perennial or seasonal allergic rhinitis, serum sickness, transfusion reactions; Dermatologic diseases: Bullous dermatitis herpetiformis, exfoliative erythroderma, mycosis fungoides, pemphigus, severe erythema multiforme (Stevens-Johnson syndrome); Endocrine disorders: Congenital adrenal hyperplasia, hypercalcemia associated with cancer, nonsuppurative thyroiditis. Synthetic analogs may be used in conjunction with mineralocorticoids where applicable; in infancy mineralocorticoid supplementation is of particular importance; Gastrointestinal diseases: During acute episodes in regional enteritis and ulcerative colitis; Hematologic disorders: Acquired (autoimmune) hemolytic anemia, Diamond-Blackfan anemia, pure red cell aplasia, selected cases of secondary thrombocytopenia; Neoplastic diseases: Palliative management of leukemias and lymphomas; Nervous system: Acute exacerbations of multiple sclerosis; cerebral edema associated with primary or metastatic brain tumor or craniotomy; Ophthalmic diseases: Sympathetic ophthalmia, temporal arteritis, uveitis and ocular inflammatory conditions unresponsive to topical corticosteroids; Renal diseases: To induce diuresis or remission of proteinuria in idiopathic nephrotic syndrome or that due to lupus erythematosus; Respiratory diseases: Berylliosis, fulminating or disseminated pulmonary tuberculosis when used concurrently with appropriate antituberculous chemotherapy, idiopathic eosinophilic pneumonias, symptomatic sarcoidosis; Rheumatic disorders: Adjunctive therapy for short-term administration in acute gout flares; acute rheumatic carditis; ankylosing spondylitis; psoriatic arthritis; rheumatoid arthritis, including juvenile rheumatoid arthritis (selected cases may require low-dose maintenance therapy); treatment of dermatomyositis, polymyositis, and systemic lupus erythematosus; Miscellaneous: Trichinosis with neurologic or myocardial involvement, tuberculous meningitis with subarachnoid block or impending block when used with appropriate antituberculous chemotherapy; Adjunctive therapy for short-term administration in acute gout flares, acute and subacute bursitis, acute nonspecific tenosynovitis, epicondylitis, rheumatoid arthritis, synovitis of osteoarthritis; Treatment of alopecia areata; discoid lupus erythematosus; keloids; localized hypertrophic, infiltrated, inflammatory lesions of granuloma annulare, lichen planus, lichen simplex chronicus (neurodermatitis), and psoriatic plaques; necrobiosis lipoidica diabeticorum; Accelerate fetal lung maturation;

Betamethasone valerate and Betamethasone diproprionate administered together: Dermatoses: Relief of inflammatory and pruritic manifestations of corticosteroid-responsive dermatoses; Dermatoses of the scalp: Relief of inflammatory and pruritic manifestations of corticosteroid-responsive dermatoses of the scalp; Plaque psoriasis (spray; patch): Treatment of mild to moderate plaque psoriasis in patients 18 years and older;

Clobetasol (Clobetasol propionate form): Steroid-responsive dermatoses;

Clobetasone (Clobetasone butyrate form): Dermatitis: Management of localized eczema and dermatitis including atopic eczema and irritant and allergic contact dermatitis;

Clocortolone (Clocortolone pivalate form): Steroid-responsive dermatoses;

Desoximetasone: Relief of inflammation and pruritic symptoms of corticosteroid-responsive dermatoses; Plaque psoriasis treatment;

Dexamethasone (Dexamethasone, dexamethasone phosphate, and dexamethasone sodium phosphate forms): Oral, IV, or IM injection: Anti-inflammatory or immunosuppressant agent in the treatment of a variety of diseases, including those of allergic, hematologic (e.g., immune thrombocytopenia), dermatologic, neoplastic, rheumatic, autoimmune, nervous system, renal, and respiratory origin; primary or secondary adrenocorticoid deficiency (not first line); management of shock, cerebral edema, and as a diagnostic agent; Intra-articular or soft tissue injection: As adjunctive therapy for short-term administration in synovitis of osteoarthritis, rheumatoid arthritis, acute and subacute bursitis, acute gouty arthritis, epicondylitis, acute nonspecific tenosynovitis, and posttraumatic osteoarthritis; Intralesional injection: Keloids; localized hypertrophic, infiltrated, inflammatory lesions of lichen planus, psoriatic plaques, granuloma annulare, and lichen simplex chronicus (neurodermatitis); discoid lupus erythematosus; necrobiosis lipoidica diabeticorum; alopecia areata; and cystic tumors of an aponeurosis or tendon (ganglia); Off-Label Use: Acute mountain sickness/high-altitude cerebral edema; Antiemetic regimens: chemotherapy-associated nausea and vomiting, prevention; Antiemetic regimens: radiation therapy-associated nausea and vomiting, prevention; Asthma, acute exacerbation; Coronavirus disease 2019 (COVID-19), treatment; Fetal lung maturation, acceleration of; Meningitis (bacterial), prevention of neurologic complications; Multiple myeloma;

Diflorasone (Diflorasone diacetate form): Dermatoses: Treatment of inflammation and pruritic symptoms of corticosteroid-responsive dermatoses;

Difluocortolone: Acute and chronic skin disease: Treatment of acute and chronic skin diseases responsive to the anti-inflammatory, antipruritic, and antiallergic effects of topical corticosteroids;

Fluticasone (Fluticasone propionate and fluticasone furoate forms): Asthma; Chronic obstructive pulmonary disease; Eosinophilic esophagitis (oral); Allergic rhinitis; Nasal polyps; Nonallergic rhinitis; Upper respiratory allergies; Acute bacterial rhinosinusitis, adjunct to antibiotics (empiric treatment); Chronic rhinosinusitis; Viral rhinosinusitis symptomatic relief; Dermatoses;

Halometasone: Treatment of steroid responsive skin disorders;

Mometasone (Mometasone furoate form): Corticosteroid-responsive dermatoses; Allergic rhinitis (seasonal and perennial); Nasal congestion associated with seasonal rhinitis; Nasal polyps; Seasonal allergic rhinitis (prophylaxis); Chronic rhinosinusitis; Rhinosinusitis, adjunctive treatment (acute); Rhinosinusitis, treatment (acute, mild to moderate, uncomplicated); Asthma;

Rimexolone: Ophthalmic inflammatory conditions: Treatment of postoperative inflammation following ocular surgery; treatment of anterior uveitis;

Amcinonide: Relief of the inflammatory and pruritic manifestations of corticosteroid-responsive dermatoses;

Budesonide: Ulcerative colitis; Allergic rhinitis; Upper respiratory symptoms: Relief of symptoms of hay fever or other upper respiratory allergies (e.g., nasal congestion, runny nose, itchy nose, sneezing); Nasal polyps; Rhinitis; Acute bacterial rhinosinusitis, adjunct to antibiotics (empiric treatment); Chronic rhinosinusitis; Asthma; Chronic obstructive pulmonary disease (acute exacerbation); Chronic obstructive pulmonary disease (stable); Eosinophilic esophagitis; Crohn disease, mild to moderate; Microscopic (lymphocytic and collagenous) colitis;

Ciclesonide: Seasonal and perennial allergic rhinitis; Acute bacterial rhinosinusitis, adjunct to antibiotics (empiric treatment); Chronic rhinosinusitis; Asthma;

Deflazacort: Duchenne muscular dystrophy;

Desonide: Atopic dermatitis; Corticosteroid-responsive dermatoses;

Flunisolide: Asthma; Rhinitis; Acute bacterial rhinosinusitis, adjunct to antibiotics (empiric treatment); Chronic rhinosinusitis; Non-allergic rhinitis; Symptomatic relief of viral rhinosinusitis;

Fluocinonide: Inflammatory and pruritic dermatologic conditions;

Halcinonide: Steroid-responsive dermatoses;

Cholesterol: Prevention/treatment of vitamin and mineral deficiencies;

Estradiol valerate: Breast cancer, metastatic; Hypoestrogenism (female); Osteoporosis prevention (female); Prostate cancer, advanced; Vasomotor symptoms associated with menopause; Vulvar and vaginal atrophy associated with menopause; Functional hypothalamic amenorrhea with low bone density (young adult females); Hormone therapy for transgender females (male-to-female);

Hydrocortisone (Hydrocortisone, hydrocortisone acetate, hydrocortisone buteprate, hydrocortisone butyrate, hydrocortisone succinate, and hydrocortisone valerate forms): Anal and genital pruritus, external; Corticosteroid-responsive dermatoses (e.g., atopic dermatitis, contact dermatitis, vulvar dermatitis, psoriasis, seborrheic dermatitis); Hemorrhoids; Ulcerative colitis; Stasis dermatitis; Vaginitis, desquamative inflammatory; Allergic states: Control of severe or incapacitating allergic conditions intractable to adequate trials of conventional treatment in drug hypersensitivity reactions, perennial or seasonal allergic rhinitis, serum sickness, transfusion reactions, or acute noninfectious laryngeal edema; Dermatologic diseases: Atopic dermatitis; bullous dermatitis herpetiformis; contact dermatitis; exfoliative dermatitis; exfoliative erythroderma; pemphigus; severe erythema multiforme (Stevens-Johnson syndrome); severe psoriasis; severe seborrheic dermatitis; mycosis fungoides; Edematous states: To induce diuresis or remission of proteinuria in the nephrotic syndrome, without uremia, of the idiopathic type or that due to lupus erythematosus; Endocrine disorders: Acute adrenocortical insufficiency; congenital adrenal hyperplasia; hypercalcemia associated with cancer; nonsuppurative thyroiditis; primary or secondary adrenocortical insufficiency; preoperatively and in the event of serious trauma or illness, in patients with known adrenal insufficiency or when adrenocortical reserve is doubtful; shock unresponsive to conventional therapy if adrenocortical insufficiency exists or is suspected; GI diseases: To tide the patient over a critical period of the disease in ulcerative colitis and regional enteritis; Hematologic disorders: Acquired (autoimmune) hemolytic anemia; congenital (erythroid) hypoplastic anemia (Diamond Blackfan anemia); erythroblastopenia (RBC anemia); immune thrombocytopenia (formerly known as idiopathic thrombocytopenic purpura) in adults; pure red cell aplasia; select cases of secondary thrombocytopenia; Neoplastic diseases: Palliative management of leukemias and lymphomas (adults); acute leukemia of childhood; Nervous system: Cerebral edema associated with primary or metastatic brain tumor, or craniotomy; Ophthalmic diseases: Severe acute and chronic allergic and inflammatory processes involving the eye, such as allergic conjunctivitis; allergic corneal marginal ulcers; anterior segment inflammation; chorioretinitis; diffuse posterior uveitis and choroiditis; herpes zoster ophthalmicus; iritis and iridocyclitis; keratitis; optic neuritis; sympathetic ophthalmia; other ocular inflammatory conditions unresponsive to topical corticosteroids; Respiratory diseases: Aspiration pneumonitis; bronchial asthma; berylliosis; fulminating or disseminated pulmonary tuberculosis when used concurrently with appropriate antituberculous chemotherapy; idiopathic eosinophilic pneumonias; Loeffler syndrome (not manageable by other means); symptomatic sarcoidosis; Rheumatic disorders: As adjunctive therapy for short-term administration in acute and subacute bursitis, acute gouty arthritis, acute nonspecific tenosynovitis, ankylosing spondylitis, epicondylitis, posttraumatic osteoarthritis, psoriatic arthritis, rheumatoid arthritis, including juvenile rheumatoid arthritis, synovitis of osteoarthritis; during an exacerbation or as maintenance therapy in acute rheumatic carditis, dermatomyositis (polymyositis), temporal arteritis, and systemic lupus erythematosus; Miscellaneous: Trichinosis with neurologic or myocardial involvement; tuberculous meningitis with subarachnoid block or impending block when used concurrently with appropriate antituberculous chemotherapy; In-hospital cardiac arrest; Septic shock; Thyroid storm;

Triamcinolone hexacetonide: Symptomatic treatment of subacute and chronic inflammatory joint diseases including: synovitis, tendinitis, bursitis, epicondylitis, rheumatoid arthritis (RA), juvenile idiopathic arthritis (JIA), osteoarthritis, or post-traumatic arthritis; and

Diflucortolone valerate: Acute and chronic skin disease.

Compounds having steroid core structures are also used in treating a wide variety of disorders in animals. For example, listed below are compounds having steroid core structures, and disorders they are used to treat in veterinary subjects, including dogs, cats, horses, swine, and cattle. In some embodiments, the composite particles described herein comprise the indicated compounds, and the disclosure provides a method of using such particles to treat the indicated disorders and/or for the following purposes in veterinary subjects:

Testosterone: Dogs—Testosterone-responsive urinary incontinence in neutered males; Dermatitis: bilateral alopecia. Cats—Testosterone-responsive urinary incontinence in neutered males;

Boldenone (Boldenone undecylenate form): Horses—as an aid for treating debilitated horses when an improvement in weight, haircoat, or general physical condition is desired;

Nandrolone: General Veterinary Patients—stimulate erythropoiesis in patients with certain anemias (e.g., secondary to renal failure, aplastic anemias);

Altrenogest: Horses—To suppress estrus or maintain pregnancy when progestin deficient. Swine—Synchronize estrus. Dogs—Luteal deficiency; prevent premature delivery;

Methyltestosterone: Female dogs—Treatment of estrogen-dependent tumors; Pseudopregnancy; Hormonal-dependent alopecias. Male dogs—Deficient libido; Testosterone-responsive incontinence; Certain hormonal alopecias. Cats—Hormonal-dependent alopecias; Increasing libido;

Stanozolol: Horses—Improve appetite, promote weight gain, and increase strength and vitality; treatment for chronic osteoarthritis. Dogs—Improve appetite, promote weight gain, and increase strength and vitality; Collapsing trachea. Cats—Improve appetite, promote weight gain, and increase strength and vitality;

Mibolerone: Female dogs—Estrus prevention;

Danazol: Dogs—Treatment of canine immune-mediated thrombocytopenia and hemolytic anemia. Cats—Autoimmune hemolytic anemia and thrombocytopenia;

Osaterone acetate: Male dogs—Benign prostatic hypertrophy;

Spironolactone: Dogs—Potassium sparing diuretic or for adjunctive treatment for heart failure;

Estradiol: Horses—Enhancing estrus behavior and receptivity in ovariectomized mare. Dogs—

Estrogen-responsive urinary incontinence; Abortifacient. Cats—Abortifacient. Cattle Abortifacient;

Megestrol (Megestrol acetate form): Female dogs—For postponement of estrus & the alleviation of false pregnancy; Male dogs—Benign prostatic hypertrophy. Cats—Many dermatologic & behavior-related conditions;

Estriol: Female dogs estrogen-responsive urinary incontinence in ovariohysterectomized female dogs;

Aglepristone: Dogs—Pregnancy termination; Pyometra complex. Cats—Progesterone-dependent mammary hyperplasia;

Medroxyprogesterone (Medroxyprogesterone acetate form): Dogs—Progestin-responsive dermatitis; aggressive behaviors; long-term reproductive control; treatment of young German shepherd dwarfs; short-term treatment of benign prostatic hypertrophy; luteal insufficiency. Cats—Sexually dimorphic behavior problems such as roaming, inter-male aggressive behaviors, spraying, and mounting; Feline psychogenic dermatitis and alopecia;

Trilostane: Dogs—Treatment of pituitary-dependent hyperadrenocorticism (PDH) and for the treatment of hyperadrenocorticism (HAC) associated with adrenocortical tumors (AT).

Cortisone (Cortisone acetate forms): Dogs—Oral treatment of hypoadrenocorticism;

Fluorometholone: General Veterinary Patients—Treatment of inflammation of the palpebral and bulbar conjunctiva, cornea, and anterior segment of the globe (blepharitis, conjunctivitis, keratitis, anterior uveitis);

Difluprednate: General Veterinary Patients—Treatment of inflammation following ocular injury or cataract surgery or to treat generalized inflammatory conditions of the anterior segment (conjunctivitis, keratitis, anterior uveitis);

Fludrocortisone (Fludrocortisone acetate form): Small Animals—Treatment of hypoadrenocorticism (Addison's disease); Adjunctive therapy in hyperkalemia;

Loteprednol: General Veterinary Patients—Treatment of inflammatory conditions of the palpebral and bulbar conjunctiva, cornea, and anterior segment of the globe (blepharitis, conjunctivitis, keratitis, anterior uveitis);

Methylprednisolone (methylprednisolone acetate and methylprednisolone succinate forms): General Veterinary Patients—Replacement of glucocorticoid activity in patients with adrenal insufficiency; Anti-inflammatory agent; Immunosuppressant;

Prednisolone/Prednisone (Treated as bioequivalents): General Veterinary Patient—Replacement or supplementation (e.g., relative adrenal insufficiency associated with septic shock) for glucocorticoid deficiency secondary to hypoadrenocorticism; Anti-inflammatory agent; Immunosuppressant; Antineoplastic agent.

Triamcinolone (Triamcinolone acetonide form): General Veterinary Patients—Focal (e.g., pedal) or multifocal lesions for relatively short durations;

Betamethasone: General Veterinary Patients—Focal (e.g., pedal) or multifocal lesions for relatively short durations; Horses—intra-articular injection for treating pain and inflamed joints. Dogs—Induce premature labor;

Dexamethasone: General Veterinary Patients—Diagnostic agent to test for hyperadrenocorticism; Replacement or supplementation (e.g., relative adrenal insufficiency associated with septic shock) for glucocorticoid deficiency secondary to hypoadrenocorticism; Anti-inflammatory agent; Immunosuppression; Antineoplastic agent;

Flumethasone: Horses—Musculoskeletal conditions due to inflammation, where permanent structural changes do not exist, such as bursitis, carpitis, osselets, and myositis; Allergic states such as urticaria (hives) and insect bites. Dogs—Musculoskeletal conditions due to inflammation of muscles or joints and accessory structures, where permanent structural changes do not exist, such as arthritis, osteoarthritis, intervertebral disc syndrome and myositis; Certain acute and chronic dermatoses of varying etiology to help control the pruritus, irritation, and inflammation associated with these conditions; Allergic states such as urticaria and insect bites. Cats—Certain acute and chronic dermatoses of varying etiology to help control the pruritus, irritation, and inflammation associated with these conditions;

Fluticasone (Fluticasone propionate form): Horses—Recurrent airway obstruction or inflammatory airway disease. Dogs—Chronic cough. Cats—Feline asthma;

Mometasone (Mometasone furoate form): General Veterinary Patient—Focal (e.g., pedal) or multifocal lesions and for relatively short durations;

Rimexolone: General Veterinary Patients—Symptomatic relief of corticosteroid-responsive inflammatory conditions of the palpebral and bulbar conjunctiva, cornea, and anterior segment of the globe (e.g., allergic conjunctivitis, acne rosacea, superficial punctate keratitis, iritis, and cyclitis). Horses—Treatment of uveitis;

Budesonide: Small Animals—Treatment of inflammatory intestinal diseases; dermatitis; Corticosteroid-responsive dermatoses;

Deoxycorticosterone: Dogs—Parenteral treatment of adrenocortical insufficiency (Addison's disease). Cats—Parenteral treatment of adrenocortical insufficiency (Addison's disease);

Alfaxalone: Dogs—Induction and maintenance of anesthesia and for induction of anesthesia followed by maintenance with an inhalant anesthetic. Cats—Induction and maintenance of anesthesia and for induction of anesthesia followed by maintenance with an inhalant anesthetic;

Hydrocortisone: General Veterinary Patient—Focal (e.g., pedal) or multifocal lesions for relatively short durations; When an acute glucocorticoid/mineralocorticoid effect is desired (e.g., acute adrenal insufficiency; critical illness-related corticosteroid insufficiency [CIRCI]); Inflammatory conjunctivitis;

Desoxycorticosterone (Desoxycorticosterone pivalate form): Dogs—Treatment of hypoadrenocorticism (Addison's disease). Cats—Treatment of hypoadrenocorticism (Addison's disease); and

Isoflupredone acetate: Horses—Anti-inflammatory & immunosuppressive effects; Swine—Anti-inflammatory & immunosuppressive effects; Cattle—Anti-inflammatory & immunosuppressive effects.

Accordingly, in one aspect, the disclosure provides a method of treating a liver disease or a peroxisomal disorder in a subject in need of treatment, comprising administering to the subject a therapeutically effective amount of a plurality of composite particles described herein, such as a pharmaceutical composition comprising a plurality of composite particles described herein (e.g., composite particles comprising a bile acid or a salt or ester thereof).

For example, in one aspect, the disclosure provides a method of treating a liver disease in a subject in need of treatment, comprising administering to the subject a therapeutically effective amount of a plurality of composite particles described herein, such as a pharmaceutical composition comprising a plurality of composite particles described herein (e.g., composite particles comprising a bile acid or a salt or ester thereof). In some embodiments, the liver disease is a bile acid synthesis disorder. In some embodiments, the liver disease is a bile acid synthesis disorder due to a single enzyme defect. For example, in some embodiments, the single enzyme defect in the bile acid synthesis disorder is a 3β-hydroxy-Δ5-C27-steroid oxidoreductase deficiency, alpha-methylacyl-CoA racemase (AMACR) deficiency, amino acid N-acyltransferase deficiency, bile acid CoA ligase deficiency, cholesterol 7α-hydroxylase deficiency, Δ4-3-oxosteroid 5β-reductase deficiency, oxysterol 7α-hydroxylase deficiency, sterol 27-hydroxylase deficiency (cerebrotendinous xanthomatosis), or trihydroxycholestanoic acid CoA oxidase deficiency. In other embodiments, the liver disease is selected from primary biliary cholangitis, primary sclerosing cholangitis, bile duct stones, and non-alcoholic fatty liver disease. In some embodiments, the liver disease is primary biliary cholangitis.

In some embodiments, the disclosure provides a method of treating a peroxisomal disorder in a subject in need of treatment, comprising administering to the subject a therapeutically effective amount of a plurality of composite particles described herein, such as a pharmaceutical composition comprising a plurality of composite particles described herein. In some embodiments, the peroxisomal disorder is a Zellweger spectrum disorder. Zellweger spectrum disorders are a group of autosomal recessive genetic disorders caused by mutations in PEX genes that encode peroxins; subdivisions of the spectrum are Zellweger syndrome, neonatal adrenoleukodystrophy, and infantile Refsum disease. In some embodiments, the subject is suffering from a Zellweger spectrum disorder with manifestations of liver disease, steatorrhea, or complications from decreased fat soluble vitamin absorption.

In some embodiments, the disclosure provides a method of treating a disorder selected from cardiometabolic disease, gallstones, type-2 diabetes, human immunodeficiency virus type 1 (HIV-1), and acute pancreatitis, comprising administering to the subject a therapeutically effective amount of a plurality of composite particles described herein, such as a pharmaceutical composition comprising a plurality of composite particles described herein (e.g., composite particles comprising a bile acid or a salt or ester thereof).

In one aspect, the disclosure provides a method of non-surgical removal of a localized fat deposit in a subject, comprising contacting the deposit with an effective amount of a plurality of composite particles described herein, such as a pharmaceutical composition comprising a plurality of composite particles described herein (e.g., composite particles comprising a bile acid or a salt or ester thereof). In some embodiments, the subject has a localized fat deposit and desires to remove the deposit. In some embodiments, the localized fat deposit is located in the submental region of the subject. In some embodiments, the localized fat deposit is located in the abdominal region of the subject. In some embodiments, the deposit is contacted with the composition by subcutaneous injection.

In an aspect, the disclosure provides a method of reducing a subcutaneous fat deposit in a subject in need thereof, comprising administering locally to the subcutaneous fat deposit in the subject an effective amount of a plurality of composite particles described herein, such as a pharmaceutical composition comprising a plurality of composite particles described herein (e.g., composite particles comprising a bile acid or a salt or ester thereof). In some embodiments, the subject has a subcutaneous fat deposit and desires to remove the deposit. In some embodiments, the subcutaneous fat deposit is located in the submental region of the subject. In some embodiments, the subcutaneous fat deposit is located in the abdominal region of the subject. In some embodiments, the deposit is contacted with the composition by subcutaneous injection. In some embodiments, the subcutaneous fat deposit is associated with a condition selected from the group consisting of obesity, fat redistribution syndrome, eyelid fat herniation, lipomas, Dercum's disease, lipodystrophy, buffalo hump lipodystrophy, dorsocervical fat, visceral adiposity, breast enlargement, hyperadiposity, diffused body fat around trunk and arms, and fat deposits associated with cellulite.

In some embodiments, the disclosure provides a method of treating cancer in a subject in need thereof, comprising administering to the subject a therapeutically effective amount of a plurality of composite particles described herein, such as a pharmaceutical composition comprising a plurality of composite particles described herein (e.g., composite particles comprising a bile acid or a salt or ester thereof). In some embodiments, the cancer is selected from colorectal cancer, cervical cancer, gastric cancer, and liver cancer. In some embodiments, the cancer is colorectal cancer.

In some embodiments, the disclosure provides a method of reducing the proliferation of cancer cells, comprising contacting the cells with an effective amount of a plurality of composite particles described herein, such as a pharmaceutical composition comprising a plurality of composite particles described herein (e.g., composite particles comprising a bile acid or a salt or ester thereof). In some embodiments, the cancer cells are selected from colorectal cancer, cervical cancer, gastric cancer, and liver cancer cells. In some embodiments, the cancer cells are colorectal cancer cells.

In some embodiments, the disclosure provides a method of treating a disorder in a subject, wherein the disorder is selected from the group consisting of endocrine disorders, rheumatic disorders, collagen diseases, dermatologic diseases, allergic states, ophthalmic diseases, respiratory diseases, hematologic disorders, neoplastic diseases, gastrointestinal diseases, nervous system disorders, inflammatory disorders, renal diseases, comprising administering to the subject a therapeutically effective amount of a plurality of composite particles described herein, such as a pharmaceutical composition comprising a plurality of composite particles described herein (e.g., composite particles comprising a corticosteroid or a salt or ester thereof).

In some embodiments, the disclosure provides a method of treating a respiratory disease in a subject in need thereof, comprising administering to the subject a therapeutically effective amount of a plurality of composite particles described herein, such as a pharmaceutical composition comprising a plurality of composite particles described herein (e.g., composite particles comprising a corticosteroid or a salt or ester thereof). In some embodiments, the respiratory disease is selected from asthma, croup, chronic obstructive pulmonary disease (COPD), bronchitis, and pneumonia (e.g., interstitial pneumonia).

The disclosed methods involve administration of an “effective amount” or a “therapeutically effective amount” of the composite particles. As used herein, both terms refer to an amount effective, at dosages and for periods of time necessary, to achieve the desired result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound (e.g., a component of the composite particles or the compositions) are outweighed by the therapeutically beneficial effects. For example, a therapeutically effective amount of the composite particles described herein may be about 1 mg/kg to about 1000 mg/kg, about 5 mg/kg to about 950 mg/kg, about 10 mg/kg to about 900 mg/kg, about 15 mg/kg to about 850 mg/kg, about 20 mg/kg to about 800 mg/kg, about 25 mg/kg to about 750 mg/kg, about 30 mg/kg to about 700 mg/kg, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 mg/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, and about 90 mg/kg to about 100 mg/kg.

The disclosed methods may further comprise a step of administering one or more additional therapeutic agents to the subject. The composite particles and the additional therapeutic agent(s) may be administered to the subject simultaneously or sequentially. In some embodiments, the additional therapeutic agent or agents may be administered in the same composition as the composite particles. In other embodiments, there may be an interval of time between administration of the additional therapeutic agent(s) and the composite particles. In some embodiments, administration of an additional therapeutic agent with the composite particles may allow lower doses of the other therapeutic agents and/or administration at less frequent intervals. When used in combination with one or more other active ingredients, the composite particles and the other active ingredients may be used in lower doses than when each is used singly.

For example, the methods may further comprise a step of administering an additional therapeutic agent to the subject, wherein the additional therapeutic agent is selected from anti-inflammatory agents, analgesics, chemotherapy agents, and bile acids or salts thereof.

Anti-inflammatory agents suitable for use with the disclosed compositions and methods can include both steroidal anti-inflammatory agents and non-steroidal anti-inflammatory agents. Suitable steroidal anti-inflammatory agents include, but are not limited to, corticosteroids such as hydrocortisone, dexamethasone, dexamethasone phosphate, beclomethasone dipropionate, clobetasol valerate, desonide, desoxymethasone, desoxycorticosterone acetate, dichlorisone, diflorasone diacetate, diflucortolone valerate, fluadrenolone, fluclarolone acetonide, fludrocortisone, fludrocortisone acetate, flumethasone pivalate, fluosinolone acetonide, fluocinonide, flucortine butylester, fluocortolone, fluprednidene (fluprednylidene)acetate, flurandrenolone, halcinonide, hydrocortisone, hydrocortisone acetate, hydrocortisone butyrate, methylprednisolone, triamcinolone, triamcinolone acetonide, cortisone, cortodoxone, flucetonide, difluorosone diacetate, fluradrenalone acetonide, medrysone, amciafel, amcinafide, betamethasone and the balance of its esters, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, and mixtures thereof. Pharmaceutically acceptable salts and esters of these agents may also be used.

Suitable non-steroidal anti-inflammatory agents include, but are not limited to: oxicams, such as piroxicam, isoxicam, tonexicam, sudoxicam, and CP-14,304; salicylates, such as salicylic acid, aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepiract, clidanac, oxepinac, and felbinac; fenamates, such as mefenamic, meclofenamic, flufenamic, niflumic, and tolfenamic acids; propionic acid derivates, such as ibuprofen, naproxen, benoxaprofen, flurbiprofen, ketoprofen, fenoprofen, fenbufen, indoprofen, pirprofen, carprofen, oxaprozin, pranoprofen, miroprofen, tioxaprofen, suprofen, alminoprofen, and tiaprofenic; and pyrazoles, such as phenybutazone, oxyphenbutazone, feprazone, azapropazone, and trimethazone; and mixtures of any thereof. Pharmaceutically acceptable salts and esters of these agents may also be used.

Analgesics may reduce discomfort due to inflammation, particularly after parenteral administration (e.g., subcutaneous injection) of a composition of the disclosure. Suitable analgesics include, but are not limited to, injectable local amine and ester anesthetics, such as lidocaine, mepivacaine, bupivacaine, procaine, chloroprocaine, etidocaine, prilocaine and tetracaine. Mixtures of these analgesics, as well as the pharmaceutically acceptable salts and esters or these agents, may also be used.

Bile acids or salts thereof may also be used in combination with the composite particles. The separately administered bile acid or salt or ester thereof may be the same as or different from the bile acid or salt or ester thereof that is present in the composite particles. Exemplary bile acids include cholic acid, deoxycholic acid, chenodeoxycholic acid, lithocholic acid, glycocholic acid, taurocholic acid, glycodeoxycholic acid, taurodeoxycholic acid, glycochenodeoxycholic acid, taurochenodeoxycholic acid, glycolithocholic acid, taurolithocholic acid, ursodeoxycholic acid, glycoursodeoxycholic acid, and tauroursodeoxycholic acid. For example, obeticholic acid is often used in combination with ursodeoxycholic acid for treatment of primary biliary cholangitis. Accordingly, in some embodiments, when the composite particles comprise obeticholic acid, the methods further comprise administration of ursodeoxycholic acid. In other embodiments, when the composite particles comprise ursodeoxycholic acid, the methods further comprise administration of obeticholic acid.

In some embodiments, the composite particles may be used in combination with a chemotherapy agent. Exemplary chemotherapy agents include those listed in the “A to Z List of Cancer Drugs” published by the National Cancer Institute.

The following examples further illustrate aspects of the disclosure but, of course, should not be construed as in any way limiting its scope.

### EXAMPLES

### Materials and Methods

All materials were purchased from Sigma-Aldrich and used as received unless indicated otherwise.

Microparticle Characterization: Microparticles were imaged with brightfield and scanning electron microscopy (SEM) (JEOL JSM-7800FLV) microscopes. Fluorescent images of the particles were acquired using an inverted Nikon Microscope and via fluorescent lamps and a TRITC/CY3 filter. For the SEM imaging, the samples were dried on a glass slide and coated with carbon and imaged with both secondary scattered and backscattered probes. Zeta potential of the microparticles was measured via dynamic light scattering (DLS) using a Malvern Zetasizer. Energy-dispersive spectroscopy (EDS) and X-ray photoelectron spectroscopy (XPS) analyses were used to characterize the elemental composition of the particles. EDS analysis was performed on the microparticles by an Oxford XMaxN 80 mm² silicon-drift energy-dispersive X-ray spectrometer. For XPS analysis, the particles were mounted on indium foil, and the XPS analysis was done via a Kratos Axis Ultra XPS machine. HPLC and proton NMR analyses were used to confirm the presence of cholate as the primary component in the structure of the particles. For these analyses, dried particles were degraded in a 50:50 mixture of acetonitrile and water and centrifuged to separate the residual gold entrapped within their structure. The supernatant was collected and analyzed with HPLC alongside a 1% standard sodium cholate solution in 50:50 acetonitrile and water. The raw HPLC data of intensity for different retention times for both the samples were plotted using Graphpad prism software. For NMR studies, the degradation product was freeze-dried with a Labcono Lyophilizer and dissolved in deuterated water and analyzed with a Varian MR400 NMR machine alongside standard sodium cholate solution. For the deoxycholate-based particles, the spectra of the degradation products and a 1% standard solution were collected in a 50:50 mixture of deuterated acetonitrile and water.

Umbilical cords were obtained under a University of Michigan Medical School Internal Review Board (IRB-MED) approved human tissue transfer protocol.

Animal studies were conducted following the National Institute of Health guidelines for the care and use of laboratory animals and approved by the Institutional Animal Care and Use Committee (IACUC) of the University of Michigan. 8-10 week old female genetically obese (B6.Cg-Lep^{ob}/J) mice purchased from The Jackson Laboratory were used. The in vivo trials were replicated for 3 animals. All the animals had the same age and the average weight of the animals in each group were matched prior to the initiation of the study.

### Example 1

### Preparation of Bile Acid Composite Microparticles with Gold

Microparticles were fabricated using the modified double emulsion solvent evaporation combined with in situ reduction of the Au(III) ion within the emulsion droplets. Briefly, sodium citrate (30 mg) and gold(III) chloride hydrate (25 mg) was dissolved in water (50 μl) and emulsified in ethyl acetate (1.0 ml) via vortexing for 30 seconds. Afterward, 2 ml of concentrated sodium cholate solution (0.75-3% w/v) was added to the first emulsion, and the mixture was vortexed for another 30 seconds. The emulsion was then added to 0.3% sodium cholate solution (10 ml) and heated for 15 minutes at 45° C. in a closed glass vial using a water bath. The emulsion was then stirred on a stir-plate at 220 rpm at room temperature for 2 hr for evaporation of the ethyl acetate. Rhodamine loaded particles were fabricated with the same protocol and adding rhodamine (2 mg) to the inner water phase. Large gold precipitates and larger particles were filtered out using a mesh filter with the pore size of 20 μm. Cholate-based particles were then collected and separated from gold nanoparticle via low-speed centrifugation at 600 rpm for 5 minutes and discarding the supernatant.

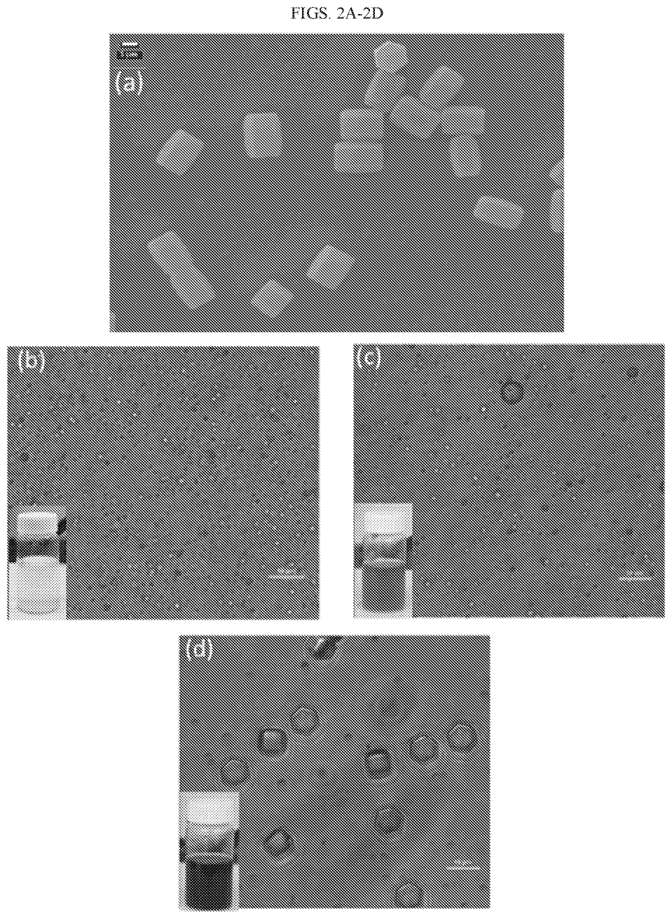

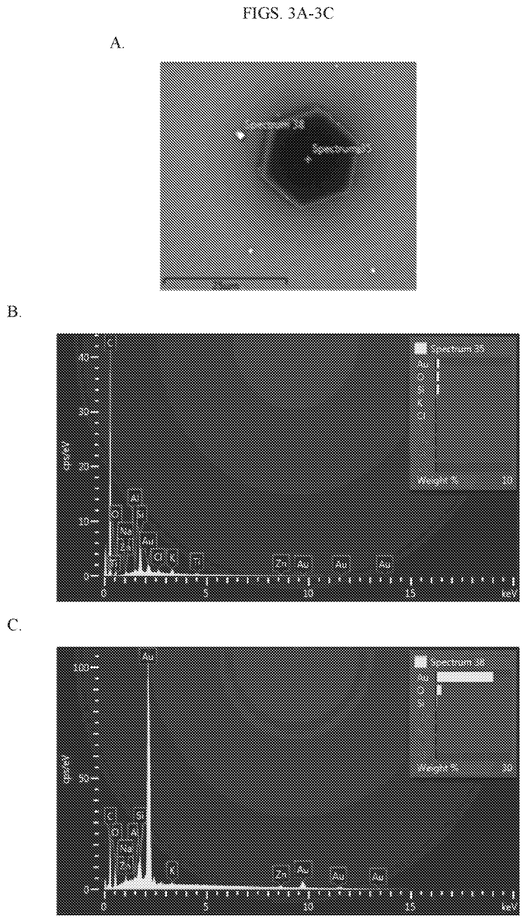

FIG. 2A shows an SEM image of the particles fabricated via this technique. FIGS. 2B-2D show brightfield microscopy images of the emulsion droplets, with insets showing images of the reaction vials, during the reaction after 0 minutes (FIG. 2B), 10 minutes (FIG. 2C), and 15 minutes (FIG. 2D) of heating. Reduction of the gold ion in the heating stage can be signaled via the color transformation of the reaction system from yellow (FIG. 2b inset) to gray (FIG. 2C inset) to dark blue-grey (FIG. 2D inset). FIGS. 2b-2d also demonstrate how the shape of the droplets changes as the self-assembly and particle formation process goes to completion. As can be seen in FIG. 2D, the self-assembly process and formation of the particles occurs only after completion of the heating step. The hexagonal microparticles are formed after the end of the 15 minutes of heating and before starting the stirring step. This demonstrates that the particle formation is a direct consequence of the in situ reduction of the Au(III) ion and its interaction with cholate.

The presence of both the gold ion and sodium cholate are crucial factors for the formation of microparticles. When gold was eliminated from the system, no particles were formed in the process. A minimum HAuCl₄ to sodium cholate mass ratio of 0.2 was required for the formation of the microparticles. Below this limit, no cholate based particles were formed, and only gold nanoparticles were recovered from the sample. The presence of an oil-water interface in the system was as well crucial to the creation of the cholate-based particles. When the organic solvent was eliminated from the system and HAuCl₄ was directly added to the sodium cholate solution, a white precipitate was immediately formed. Considering that cholic acid is a water-insoluble white powder, and the fact that HAuCl₄ is an acidic compound, the direct addition of these two compounds leads to a significant drop in the pH of the system and results in the formation of insoluble cholic acid, which could inhibit the formation of the microparticles. The existence of an organic solvent prevents this issue and enables the interaction of gold and cholate at the oil-water interface and fabrication of cholate-based solid microparticles.

The zeta potential of the particles was −66.3±3.3 mV. Others have previously shown that adsorption of bile salts on the surface of nanoparticles significantly increases the absolute negative value of their zeta potential (Macierzanka et al. Soft Matter 7, 8077-8084 (2011)). The observed highly negative zeta potential of the particles is thus consistent with cholate being the main component of the surface of the microparticles.

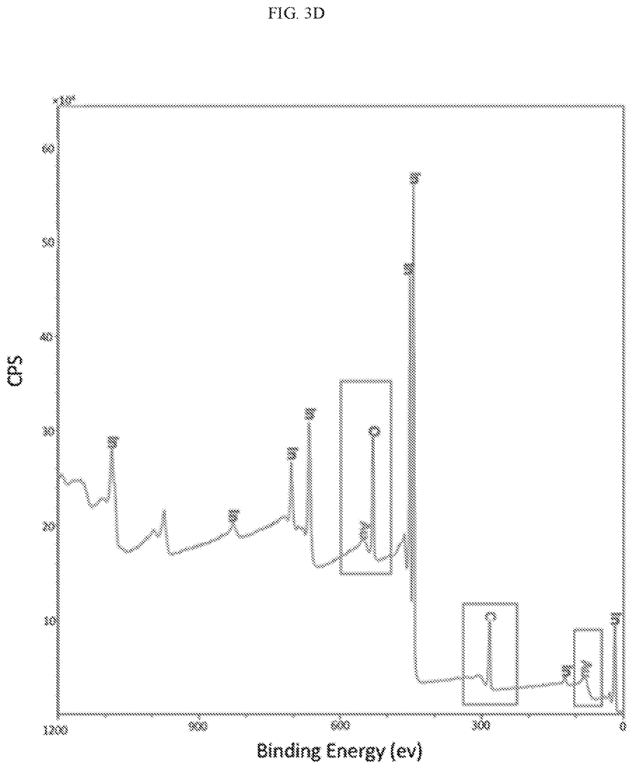

Energy-dispersive spectroscopy (EDS) was used to identify the elements incorporated into the structure of the hexagons. Elemental analysis was performed on the SEM image of the sample (FIG. 3A) after completion of the fabrication process and before any further steps for separation of the hexagons and gold nanoparticles. Carbon and gold were the main elements detected on the surface of the hexagons (FIG. 3B). The spectrum of the hexagons can be compared to the spectrum of the individual standing gold nanoparticles, which shows gold as the primary element in their composition as opposed to hexagons which are primarily composed of carbon (FIG. 3C). X-ray photoelectron spectroscopy (XPS) was used as the secondary method to confirm the elemental composition of the hexagonal microparticles. After separating gold nanoparticles from the hexagons via centrifugation, the pellet containing the hexagons was dried and mounted on indium (In) foil for XPS analysis. As shown in FIG. 3D, the sample is primarily composed of carbon and oxygen, and gold.

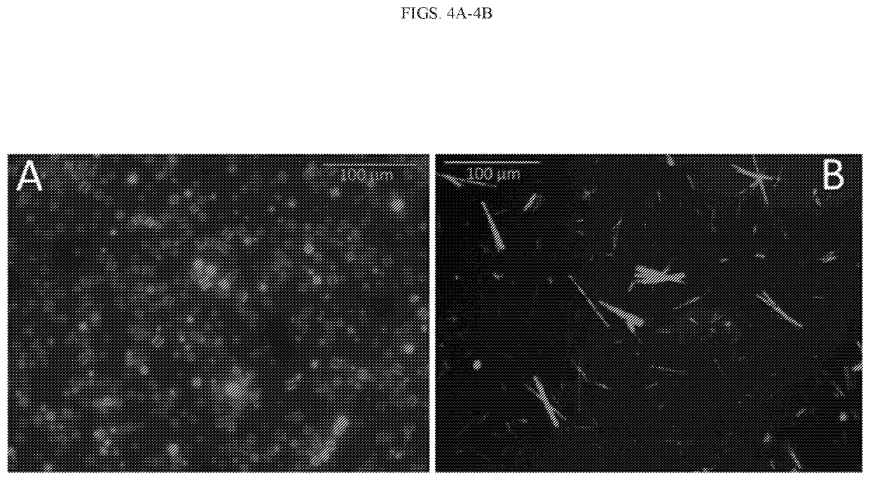

Fluorescent particles were also successfully fabricated by adding rhodamine to the inner water phase. FIG. 4A shows a fluorescence image of rhodamine-loaded cholate-based particles, and FIG. 4B shows a fluorescence image of rhodamine-loaded deoxycholate-based particles. This demonstrates the capability of the method to load hydrophilic agents within the cholate-based microparticles for combined therapy.

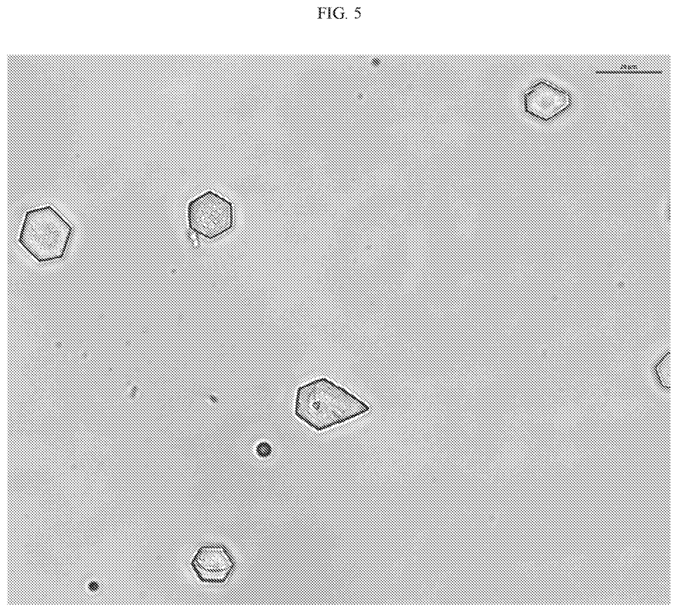

The choice of the solvent did not seem to affect the self-assembly of the droplets, and the hexagons were still formed while using dichloromethane as the solvent in the system (FIG. 5).

Deoxycholate particles were fabricated with the same procedure as the cholate-based particles and substituting sodium cholate with sodium deoxycholate in the fabrication protocol. With the same conditions used, deoxycholate-based particles have a rod-shaped morphology. A concentration of 1% sodium deoxycholate in the outer water phase resulted in an average length of 8.4±3.2 μm and width of 870±300 nm. FIG. 6A shows a brightfield microscopy image of the deoxycholate-based composite particles fabricated via this technique. FIG. 6B shows an SEM image of the deoxycholate-based composite particles (scale bar 1 μm).

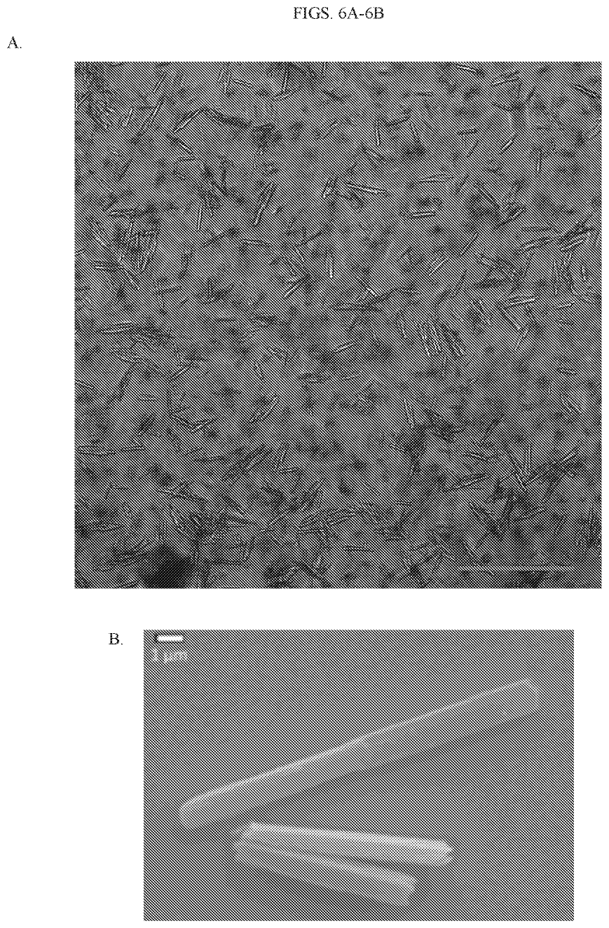

Ursodeoxycholate particles were also fabricated with the same procedure as the cholate-based particles and substituting sodium cholate with a mixture of ursodeoxycholic acid and sodium hydroxide in the fabrication protocol. The ursodeoxycholate particles are shaped as irregular hexagonal sheets, with approximate lengths of 30 μm for the large side and 11 μm for the small side. FIG. 7 shows an SEM image of the ursodeoxycholate-based composite particles fabricated via this technique (scale bar 10 μm).

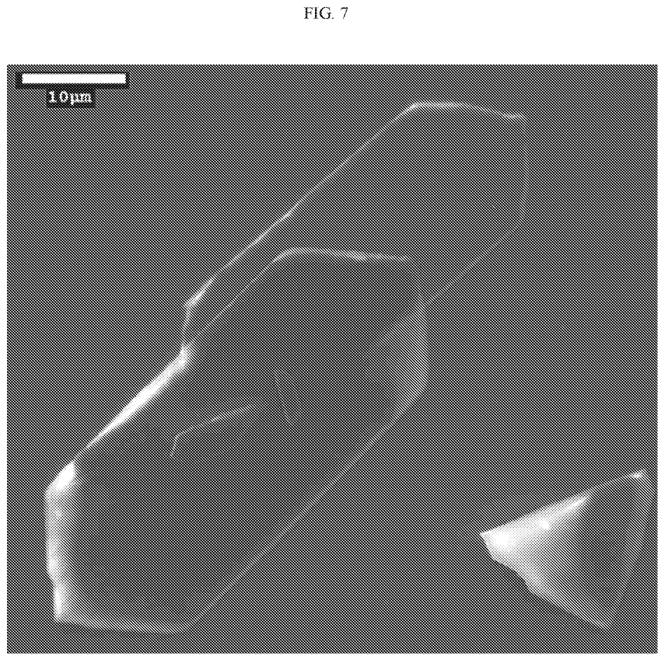

Chenodeoxycholate particles were also fabricated with the same procedure and substituting sodium cholate with sodium chenodeoxycholate, and using a lower amount of sodium citrate (1 mg or 5 mg). The chenodeoxycholate particles are shaped as spheres with approximate dimensions of 1-10 μm (scale bar 1 μm). FIG. 8A shows an SEM image of the chenodeoxycholate-based composite particles fabricated by this technique. FIG. 8B shows an EDS spectrum of the particles. The EDS readouts require a higher beam strength to get adequate readings, and the glass slide beneath the sample accounts for the Si, Al, Na, Zn, K, and Ti peaks. Nevertheless, the particles include the chenodeoxycholate salt as evidenced by the C and O peaks, and the presence of the Au peak confirms that the particles include the gold nanoparticle templates.

### Example 2

### Degradation and Release Assays

For microparticle release and degradation assays, particles were first freeze-dried with a Labcono Lyophilizer.

For rhodamine release assays, dried particles were resuspended in 1×PBS in a concentration of 5 mg/mL and rotated on an end-to-end rotator at 37° C. At different time points, the suspension was spun-down and released rhodamine was quantified via fluorescent measurement using a plate reader and the respective excitation and emission wavelengths of 553 nm and 627 nm.

For degradation studies, dried particles were resuspended in deionized water at the concentration of 10⁶ particles/ml and rotated on an end-to-end rotator at 37° C. At different desired time points, a droplet of the particle suspension was taken and dried on a glass slide and imaged via SEM to visualize their surface morphology. The cholate release assays were performed via the same protocol. At the desired time points, the particle suspension was spun-down, and the supernatant was collected. The amount of the released cholate was measured using HPLC and via quantification of the area under the curve and comparing it to a calibration curve.

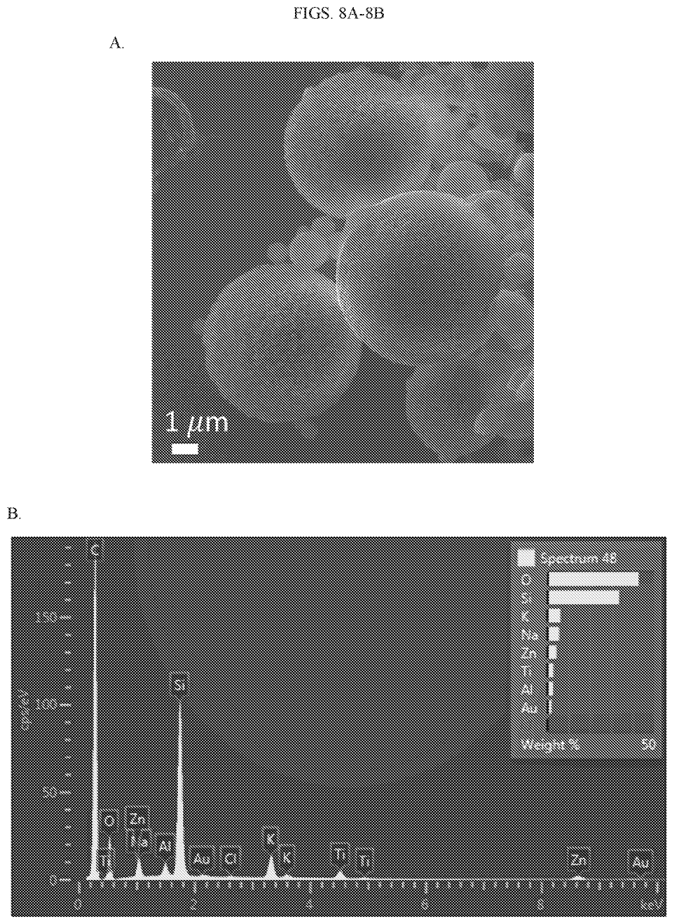

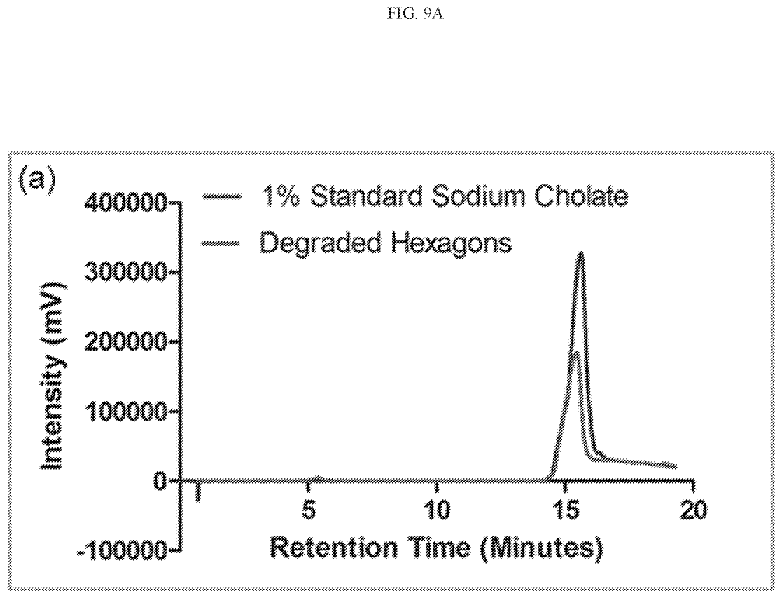

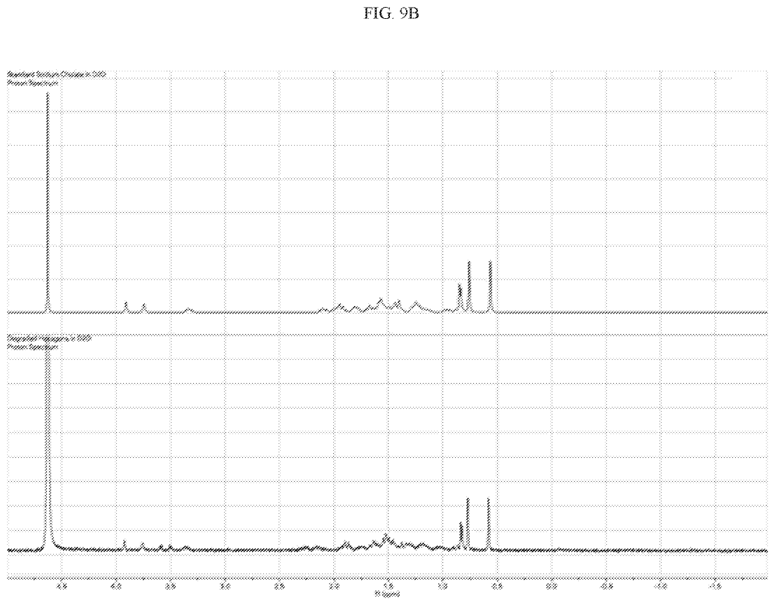

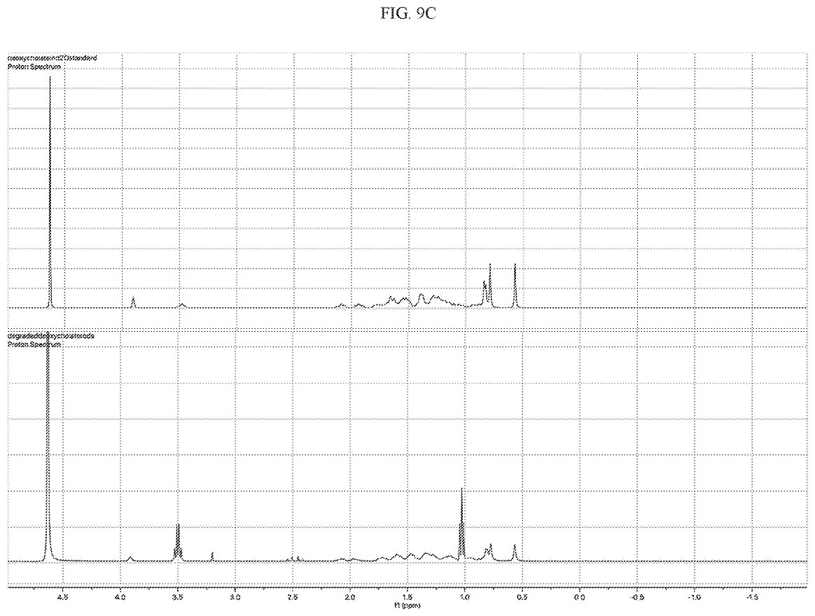

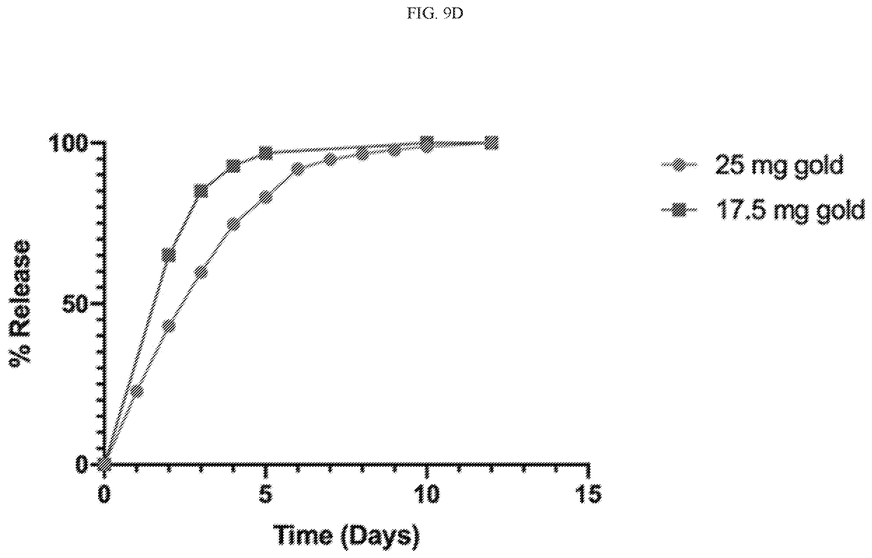

As shown in FIG. 9A, the HPLC peak for the degraded cholate composite particle hexagons (red peak) shows up at the same retention time as the standard sodium cholate (purple peak) solution. FIG. 9B shows proton NMR spectra of standard 3% sodium cholate solution and the degradation products of the cholate composite particle hexagons in deuterated water. As shown in the spectra, the peaks for both the standard and the degradation products of the particles showed up at the same chemical shift values with the same splitting pattern and the same relative intensities. These data all confirm the presence of sodium cholate as the main compound in the structure of the hexagons.

FIG. 9C shows the NMR spectrum of the degradation products of the deoxycholate-based particles in deuterated water. The dried particles were extensively sonicated to be partially degraded and centrifuged. The supernatant including the degradation product was analyzed via ¹H-NMR alongside the standard deoxycholate particles. All the peaks of the standard deoxycholate solution are present for the deoxycholate-based particles. The extra peaks present in the spectrum of the deoxycholate particles are probably the consequence of the protonation of the deoxycholate and the impurities from the fabrication/degradation process including residual citrate.

When the release profile of rhodamine from the deoxycholate-rhodamine composite particles was investigated in PBS at 37° C., up to day 5 there was a nearly linear release profile of rhodamine (FIG. 9D). This is opposite of the observed initial burst release of the hydrophilic drugs from polymeric biodegradable particles when diffusion-controlled release is dominant (Allison, Expert Opin. Drug Deliv. 5, 615-628 (2008)). Interestingly, increasing the amount of gold ion in the precursor, which may slow down their degradation due to more crosslinking, delayed the release profile of rhodamine from these particles. This shows that by adjusting the amount of gold ion in the inner water phase, the release of drugs from bile salt particles can be tuned.

### Example 3

### Degradation Kinetics

Particles were first freeze-dried with a Labcono Lyophilizer. Dried particles were then resuspended in deionized water at the concentration of 10⁶ particles/ml and rotated on an end-to-end rotator at 37° C. At different desired time points, a droplet of the particle suspension was taken and dried on a glass slide and imaged via SEM to visualize their surface morphology.

As shown in FIG. 10A, the cholate composite particles (prepared as described in Example 1) maintained their shape after being freeze dried. However, some wrinkles were observed on their surface which is due to the evaporation of water from their structure. After two hours of incubation, the wrinkles on the surface of the particles disappeared as a result of the reabsorption of water. One day of incubation started to erode the particles and continuing the incubation led to a decrease in their overall size. Incubating particles for two weeks resulted in the formation of cracks on their surface. After four weeks of incubation, all particles were broken into smaller pieces. As seen in FIG. 10A, surface erosion is the dominant mechanism for the degradation of the particles. Quantification of the released cholate content after degradation for different time points demonstrated a nearly linear release which is consistent when surface erosion is the dominant mechanism controlling degradation of the particles (von Burrkersroda et al. Biomaterials, 2002, 23, 4221)—see FIG. 10B. The results confirmed the release of 50 μg cholate/day to the water phase by degrading 1.0 mg of the dried particles.

### Example 4

### Fabrication of Microparticles with Different Sizes and Morphologies

Particles were prepared in accordance with the procedures of Example 1, but the sodium cholate concentration in the outer water phase was varied to make particles of different sizes. Table 1 shows how the size of the hexagons varies as a function of changing surfactant concentration (0.75% w/v, 2% w/v, and 3% w/v). The higher surfactant concentrations resulted in smaller particles. FIGS. 11A-11C show SEM images of the particles with different sizes ranging from diagonal of 9-3 μm fabricated via this technique; FIG. 11A shows particles prepared using 0.75% w/v sodium cholate in the outer water phase, FIG. 11B shows particles prepared using 2% w/v sodium cholate in the outer water phase, and FIG. 11C shows particles prepared using 3% w/v sodium cholate in the outer water phase. The scale bars are all 1 μm.

TABLE 1

| Sodium cholate concentration | Hexagon | Hexagon |
|---|---|---|
| in the outer water phase (w/v) | Diagonal (μm) | Height (μm) |
| 0.75% | 8.7 ± 2.3 μm | 5.3 ± 0.8 μm |
| 2% | 5.4 ± 1.1 μm | 4.3 ± 0.7 μm |
| 3% | 2.9 ± 0.3 μm | 3.6 ± 0.7 μm |

Concentrations lower than 0.75% seemed to be unable to stabilize the emulsion and resulted in particles with unsmooth surface morphologies (see FIGS. 11D-11E—FIG. 11D shows an SEM image of particles prepared using 0.5% sodium cholate in the outer water phase, and FIG. 11E shows an SEM image of particles prepared using 10% sodium cholate in the outer water phase). When sodium cholate concentrations of higher than 3% were used, particles with fibrous morphology were formed. Increasing the surfactant concentration increased the percentage of the fibers in the product. At the sodium cholate concentration of 10%, all the formed particles were rod-shaped, and no hexagons were observed in the product.

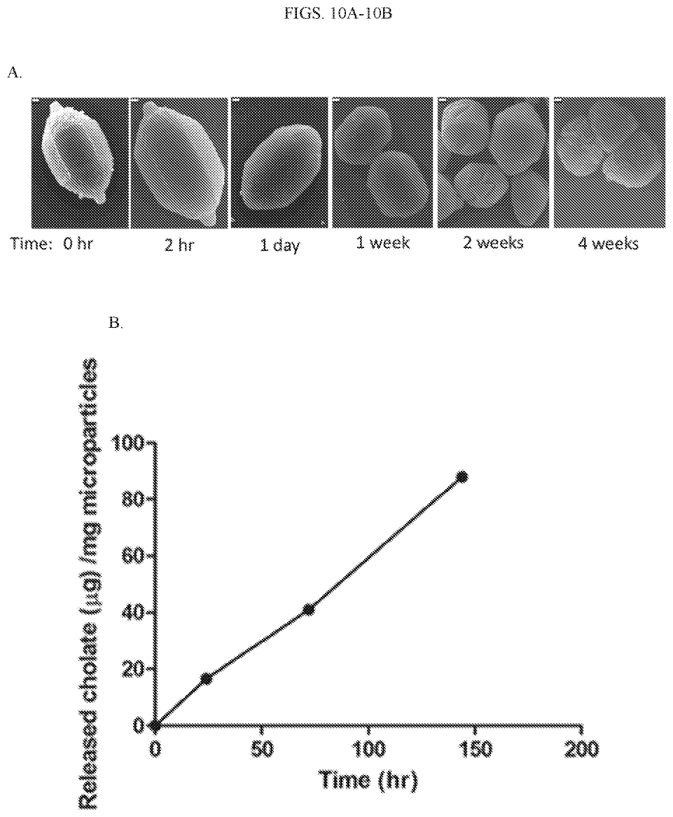

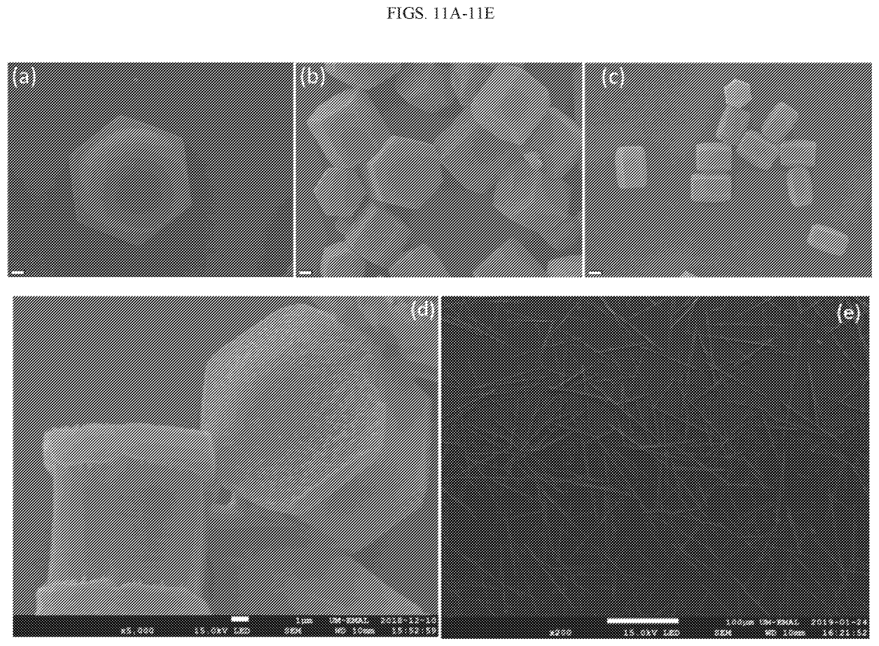

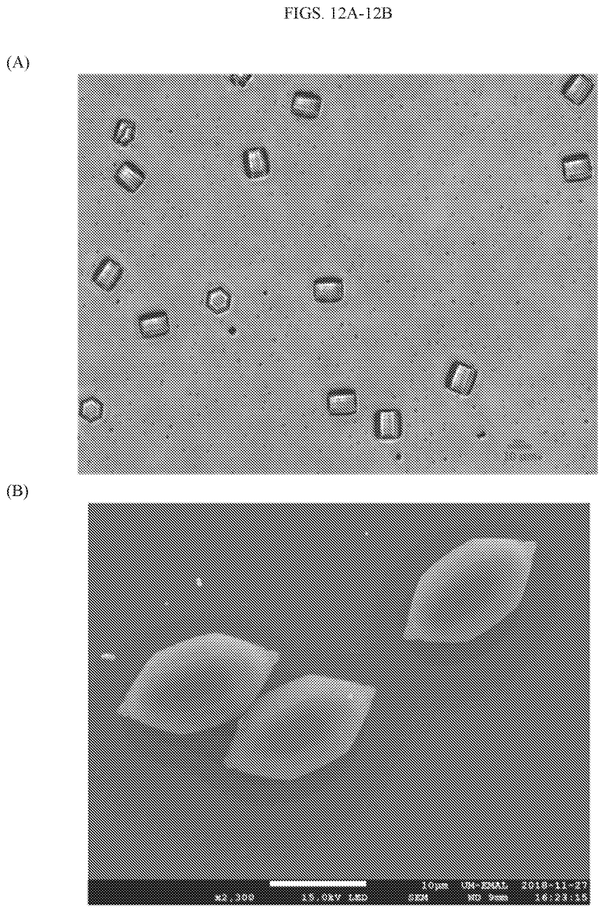

Elongated hexagonal bipyramidal cholate-based particles were fabricated with the same protocol as specified above and adding 100 μg of 6-carboxyfluorescein to the gold precursor in the inner water phase. When the shape of the microstructures was checked after the heating step, they were in the regular hexagon shape as previously described (FIG. 12A). This suggests that the presence of the dye does not interfere with the self-assembly during the heating stage. However, once the reaction mixture was stirred to evaporate the residual solvent, the unsolidified hexagonal microstructures were stretched into elongated hexagonal bipyramids as shown in FIG. 12B. This observation is most probably a consequence of the coupling of the self-assembly process and droplet dynamics. Stretching of the emulsion droplets is controlled by a dimensionless ratio called capillary number, the ratio of the viscous forces to the capillary forces, shown the following equation:

$$( C a = \frac{γ \cdot {η}_{s} \cdot a}{Γ} )$$

(γ is the shear rate, ηₛ is the water phase viscosity, α is the droplet radius, and Γ is the interfacial tension between the oil phase and the water phase; see Heslinga et al. J. Control. Release, 2009, 138, 235).

The higher capillary numbers in the system will favor the elongation of the emulsion droplets. The presence of the hydrophilic dye in the water phase and its diffusion lowers the interfacial tension of the system. The Presumptive decrease in the interfacial tension of the system by the presence of the 6-carboxyfluorescein will lead to an increase in the capillary number of the system and enable stretching of the unsolidified particles.

### Example 5

### Cell Lysis Assays

Human umbilical vein endothelial cells (HUVECs) were cultured into 12-well plates which were pre-treated with gelatin, glutaraldehyde, and glycine as previously described (Charoenphol et al. Biomaterials. 2010; 31(6):1392-1402). After reaching confluency, the cells were incubated with 1 ml of either the deoxycholate or cholate solutions of different concentrations or the suspension of the gold-cholate composite particles of known concentrations for different time-points at 37° C. and 5% CO₂. After the desired time-point, the treatment solution/suspension was removed from the wells, the cells were washed with warm 1X phosphate buffered saline (PBS) and incubated with 1 ml of the 1:25 dilution of MTS assay cell titer (Promega, WI) in 1×PBS at 37° C. and 5% CO₂ for 2 hr until the appearance of the orange color in the untreated control wells. The absorbance was then measured at 490 nm, and each condition was repeated in triplicate. The percentage of the cell viability was quantified via subtracting the background cell titer absorbance from the absorbance of the desired point and dividing it by the average signal of the untreated cells after subtracting the background.

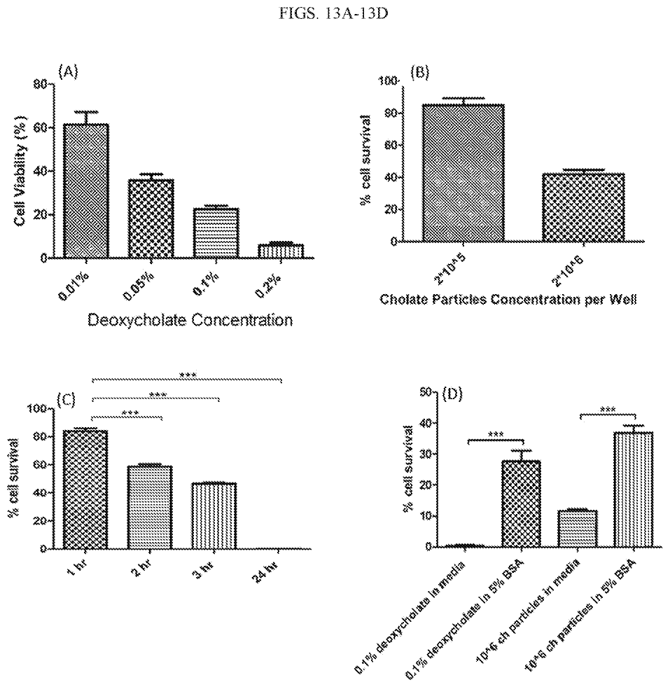

Results demonstrated the death of the HUVECs via their incubation with deoxycholate solution in media in a concentration-dependent matter (FIG. 13A). Even 0.01% of deoxycholate solution is enough to kill nearly 40% of the cells within 1 hr. Increasing the salt concentration will increase cell death. 0.2% salt solution will kill approximately 95% of the cells, and higher concentrations are enough to lyse all the cells within 1 hr. Incubating the cells with cholate-based composite particles was also able to kill the cells (FIG. 13B). Within 1 hr of incubation, 2×10⁵ particles lysed about 20% of the cells, and that number was increased to 60% when the concentration of the particles were increased 10-times to 2×10⁶ particles/well. If all the particles were degraded, the maximum concentration of the salt would approximately be 0.0015% for 2×10⁵ particles and 0.015% for 2×10⁶ particles. These numbers are assuming a well-mixed solution. However, due to the gravity, the local concentration of the particles will be higher near the cells, which makes them more potent in killing the cells.

The observed cell-lysis of the HUVECs after incubation with cholate particles was time dependent as well. As shown in FIG. 13C, when the cells were treated with 10⁶ particles/well (0.007% maximum concentration if all the particles were degraded, and the solution was well-mixed). About 20% of the cells died after 1 hr of incubation with the particles. This number was increased to 40% and 55% for the subsequent 2 hr and 3 hr time-points, and nearly 100% of the cells were died after increasing the incubation time to 24 hr.

Previous studies had reported the inhibition of the deoxycholate-induced cell lysis in the presence of albumin (Thuangtong et al. Dermatologic Surg. 2010; 36(6):899-908). The selective lysis of the fat tissue is attributed to this effect since it is not exposed to high concentrations of albumin in contrast to other tissues. To confirm that the cell-lysis induced by the particles is a result of the same cell-lysis mechanism observed for the deoxycholate, cell-lysis was investigated via incubating the cells with sodium deoxycholate and composite particles in 5% BSA (bovine serum albumin) solutions and compared it with the trend observed in culture media. As shown in FIG. 13D, an increase in the cell viability was observed in the presence of BSA for both sodium deoxycholate and cholate-based composite microparticles. Exposure of the cells to the 0.1% deoxycholate salt in media for 3 hr resulted in the death of nearly all the cells while after repeating the procedure in 5% BSA around 25% of the cells were still alive after 3 hr which is consistent with the previous literature. When the cells were exposed to 10⁶ composite cholate particles, only 10% of the cells were alive in media, and this number was increased to 35% in 5% BSA. This observation demonstrates that the albumin lysis inhibition effect observed for the salts is present for the composite microparticles.

### Example 6

### Additional Lysis Assays

For the tissue lysis assays, a known mass of the tissue was incubated with either of the PBS, salt solutions, or the particle suspensions at 37° C. for known time-points. Thereafter, the turbidity of the solution was measured by measuring the absorbance at 660 nm, and the appearance of the tissue was visualized. The amount of the released fatty acids in the mixture was quantified via a free fatty acid quantification kit purchased from Cayman Chemicals (Ann Arbor, Mich.).

The capability of the particle formulation to kill fat cells was tested by incubating them with primary subcutaneous human adipocytes. The primary subcutaneous human adipocytes cultured in 96-well plates were purchased from Zen-Bio (Research Triangle, NC). Upon arrival, 150 μl of the media was removed from each well, and the cells were incubated at 37° C. and 5% CO₂. For the lysis assays, 150 μl of the salt solution or particle suspension of the known concentration in FBS free RPMI-medium were incubated for a known time-point. Afterward, the treatment solution was aspirated, the cells were washed with warm PBS, and 150 μl of a 1:25 dilution in 1×PBS of MTS assay cell titer (purchased from Promega Corporation, Madison, Wis.) was added to each well. Plates were incubated at 37° C. and 5% CO₂ for 3 hr until the appearance of the orange color in the untreated control wells and the absorbance was measured at 490 nm. The cell viability was quantified via subtracting the background cell titer absorbance from the absorbance of the desired point and dividing it by the average signal of the untreated cells after subtracting the background.

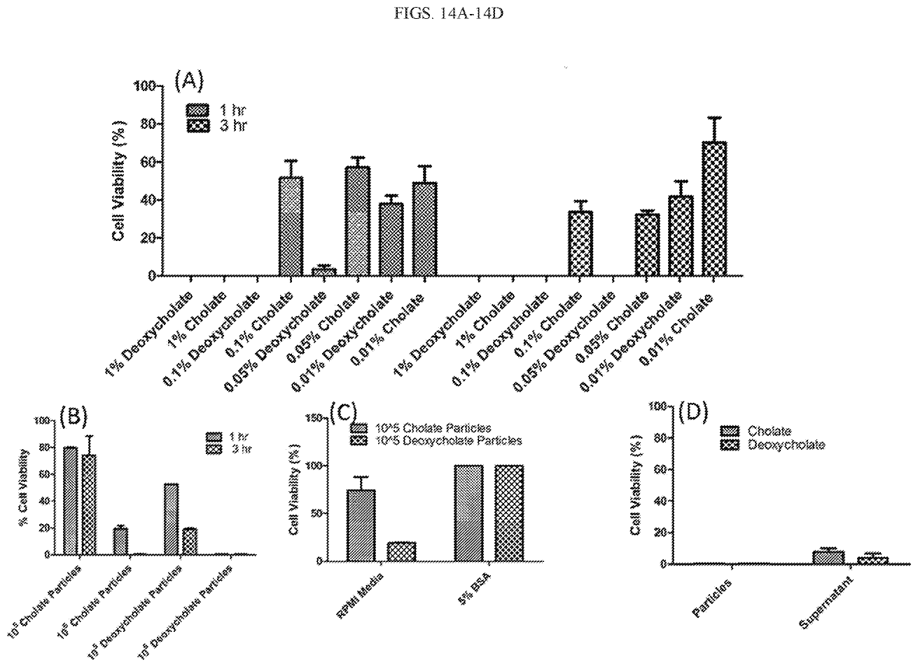

For the control experiments, the cells were incubated with different concentrations of sodium cholate and sodium deoxycholate in RPMI media. As demonstrated in FIG. 14A, sodium deoxycholate was more potent in lysing the fat cells. The deoxycholate concentrations of higher than 0.05% were enough to kill the fat cells within 1 hr. For sodium cholate, even though 1% solution was able to kill all the cells, still 60% of the cells were viable after incubation with 0.1% sodium cholate for 1 hr. The difference between the cell viabilities was not significant for the 0.1, 0.05, and 0.01% cholate concentration. Though the cell viability after incubation with 0.1% and 0.05% cholate respectively decreased from 50%, and 57% to 30% after increasing the incubation time from 1 hr to 3 hr, the general response of the cells to the salt incubation was not heavily time-dependent.

As seen with the HUVECs, both the composite cholate and deoxycholate-based particles were able to successfully lyse the adipocytes (FIG. 14B). The same as the salt solutions, deoxycholate particles were more potent in killing the cells compared to the cholate particles. In contrast to the salt solutions, the lysis response of the adipocytes to the composite particles was heavily time-dependent. When 10⁵ cholate-based particles were added to each well (the maximum concentration of 0.004% if all the particles were degraded), the particles were able to only kill about 20% of the cells independent of the incubation time. When the concentration of the particles was 10-times increased to 10⁶ particles/well (maximum effective concentration of 0.04%), 80% of the cells were killed after 1 hr of incubation, and this number was increased to nearly 100% when the incubation time was increased to 3 hr. For the deoxycholate particles, even 10⁵ particles (maximum effective concentration of 0.0035%) was enough to lyse 40% of the cells within 1 hr of incubation and 80% of them after 3 hr. For 10⁶ deoxycholate particles added per well (the maximum concentration of 0.035%), even 1 hr of incubation was enough to kill all the cells.

To investigate the lysis inhibiting effect of BSA, 10⁵ of both the cholate and the deoxycholate particles were incubated with each well of the cells for 3 hr in both media and 5% BSA solution. Though the particles were able to lyse the cells in media, the lysis effect was inhibited entirely in a 5% BSA solution (FIG. 14C). This observed result confirms the previously observed inhibition of lysis using BSA, which confirms that the particles lyse the fat cells with the same mechanism as the salt.

To show that the released cholate/deoxycholate are the active ingredients that induce the cell lysis, the particles were preincubated in media at 37° C. for 3 hr and, centrifuged the mixture to separate the undegraded particles and gold nanoparticles and incubated the supernatant with the cells. As demonstrated in FIG. 14D, the supernatant containing the released medium was able to successfully lyse 90-95% of the cells within 3 hr. The results show that the composite microparticles can gradually degrade and release cholate/deoxycholate to lyse the fat cells.

### Example 7

### Ex Vivo Adipose Tissue Lysis

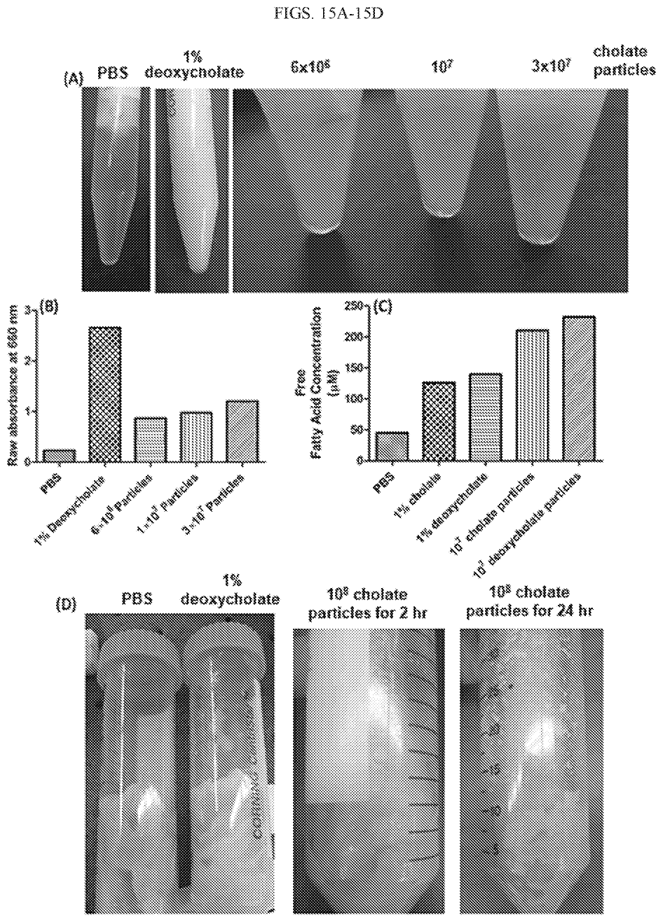

To examine the capability of the cholate and deoxycholate-based particles to lyse fat tissue, beef adipose tissue was incubated with either PBS, deoxycholate solution, or the composite particles at 37° C. for different time-points. As shown in FIG. 15A, even though incubation with PBS will have minimal effect on the solution while incubating the samples with 1% sodium deoxycholate or different numbers of the particles will start lysing the fat and resulting in a milky solution. When the turbidity of the solution was measured, is was shown that increasing the concentration of the particles will increase the absorbance of the solution which is significantly higher than the control PBS (FIG. 15B). When the amount of the free fatty acids in the incubation medium was measured there was a significant increase in the released fatty acids after incubating the particles with either of the cholate or deoxycholate salt solutions or the composite particles from 44 μM for PBS incubation to 100 μM or higher for either of the salt/particle incubations. Both of the deoxycholate salts and particles released more fatty acids compared to the cholate control of the same concentration (FIG. 15C). Incubating chicken breast with either of the salts or particle concentrations induced the release of fat and destroying of the tissue (FIG. 15D).

### Example 8

### In Vivo Lysis of Mouse Adipose Tissue

In vivo lipolysis assays were performed via subcutaneous injection of rhodamine-loaded deoxycholate microparticles into the inguinal fat pads of genetically obese mice alongside the salt solution and vehicle control. Genetically obese mice were anesthetized using isoflurane, shaved, and subcutaneously injected with 100 μL of the suspension of rhodamine-loaded deoxycholate particles or the solution of deoxycholate salt in saline (25 mg/mL) or vehicle control into their right inguinal fat pad. 100 μL of pure saline was injected into the left fat pad of the animals as the control. The weight and appearance of the animals was tracked over the course of two weeks. One group of the animals received a second dosage of particles on Day 7. After 14 days, the animals were euthanized, the right and left fat pad of the animals were removed, weighed, and fixed in 10% formalin solution overnight. Histology slides of the samples were prepared via paraffin embedding and standard hematoxylin and eosin staining. The histology slides were analyzed by blindfolded physicians and the digital scans were analyzed using QuPath software.

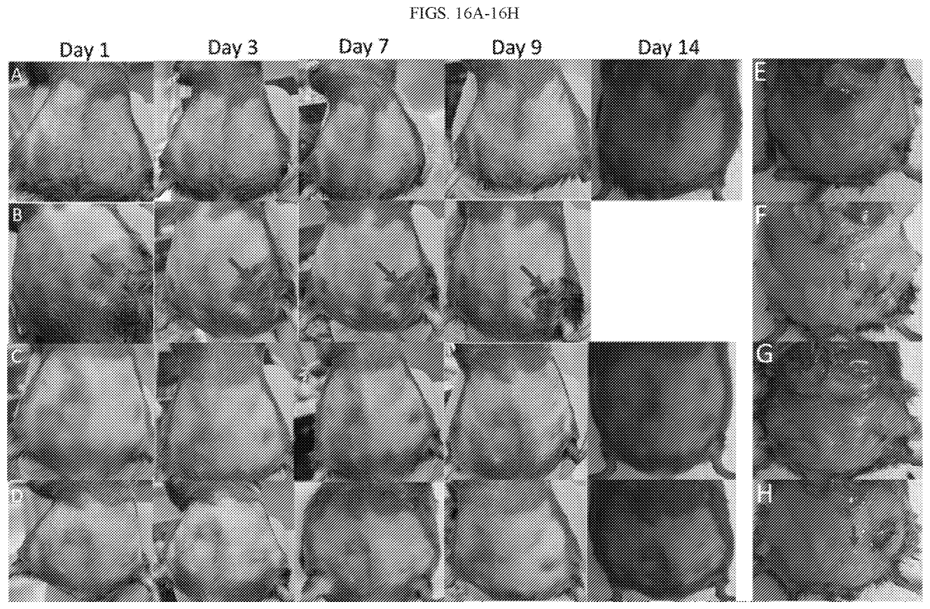

Injection of pure saline as the vehicle control did not induce any inflammation (FIG. 16A). However, severe bruising and inflammation was observed at the injection site in the animals that received sodium deoxycholate solution (FIG. 16B). 7-9 days post-injection, an ulcer developed in all of the animals that received the salt injection. These animals were euthanized immediately. However, there were no visual traces of skin inflammation and bruising in animals that received either one or two dosages of the deoxycholate composite particles (FIGS. 16C and 16D). These results show that the reported bruising and inflammation associated with the deoxycholate solution is not observable for the bile salt microparticles.

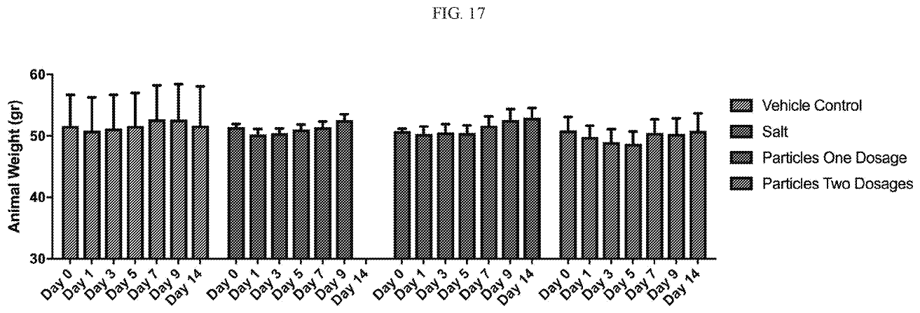

When animals were euthanized 14 days post-injection, visual evidence of local fat loss was observed in the animals that received both salt and particle injections. Clear sections of fat visible in the control (left) fat pad of animals were missing in their test (right) fat pad in the proximity of the injection site (FIGS. 16E-16H). No such difference was observed between the right and the left fat pad of the animals that had received the vehicle control (FIG. 16E). The visual difference between the right and left fat pad of animals was more prominent in the animals that had received two dosages of particles. Particles were still present in the injection site two weeks post-injection. This confirmed the gradual degradation of particles at the injection site, which promoted lipolysis while avoiding severe inflammation. No significant change in the weight of the animals was observed during the lipolytic treatment for test or vehicle control groups (FIG. 17). These results confirmed the localized lipolytic effect of the particles, consistent with what is seen in clinically used salt formulations.

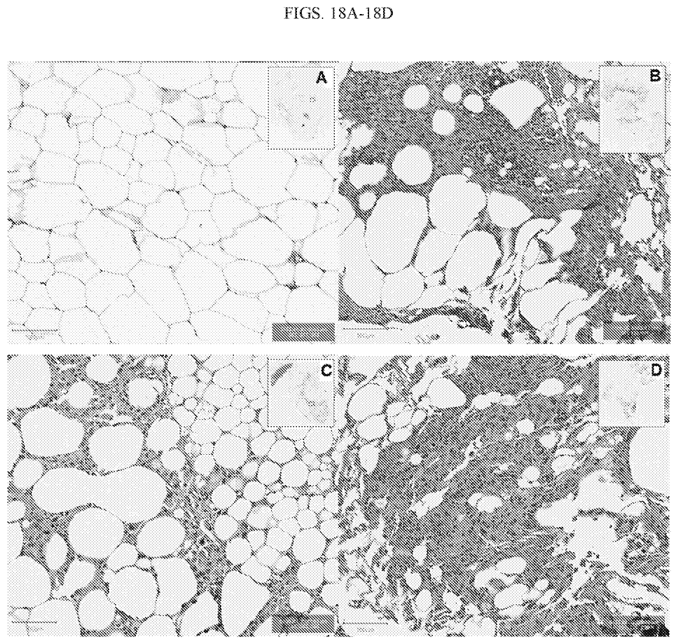

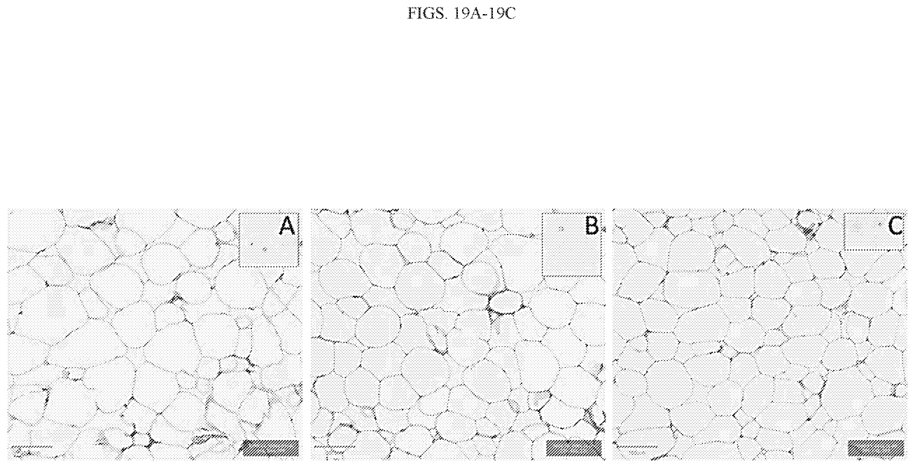

Histology sections of the fat pad also evidenced significant lipolysis in animals that had received lipolytic treatments. Crown-like structures and significant leukocyte infiltration, which are evidence of fat lysis, were observed in the right fat pad of the animals that received the salt or particle treatments (FIGS. 18A-18D). However, lipolytic evidence was not visible in the left fat pad of those animals or in the right fat pad of the animals that had received the vehicle control (FIG. 19 and FIG. 18A). This confirms that the lipolysis is a direct consequence of the local injection of the salt or particles. The results in this section confirm that the composite microparticles are able to induce localized lipolysis in the adipose tissue in vivo in the same manner as deoxycholate salt. However, the severe inflammation and ulceration reported as the side effect of sodium deoxycholate solution (Kybella) can be reduced via the utilization of composite microparticles.

### Example 9

### Cancer Cell Lysis Assays

HCT-116 colon cancer cells (ATCC® CCL247™) were purchased from American Type Culture Collection (ATCC) and cultured in 24-well plates until reaching confluency. Each well was then incubated with a specified concentration of cholate microparticles (10⁵-10⁷ particles/well) in 500 μl of RMPI media for specified time-points. After the end of the incubation time, the cells were immediately imaged using an Inverted Nikon Microscope. Afterwards, all the wells were washed with warm PBS and their viability was quantified using MTS assay. Briefly, 500 μl of 1:25 dilution of MTS assay cell titer purchased from Promega Corporation (Madison, Wis.) in 1×PBS was added to each well and incubated at 37° C. and 5% CO₂ until the appearance of the orange color in the untreated control well, and the absorbance was measured at 490 nm. The percentage of the cell viability was quantified via subtracting the background cell titer absorbance from the absorbance of the desired point and dividing it by the average signal of the untreated cells after subtracting the background.

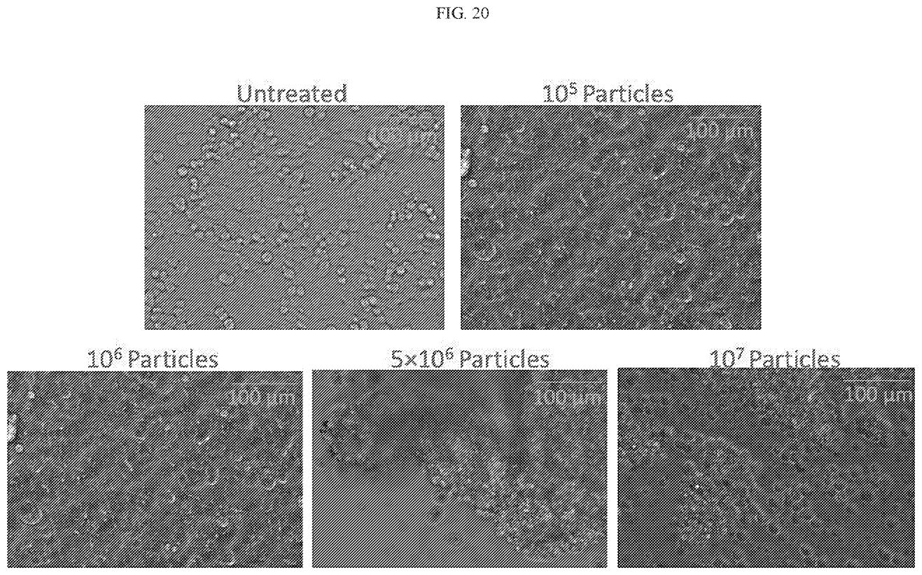

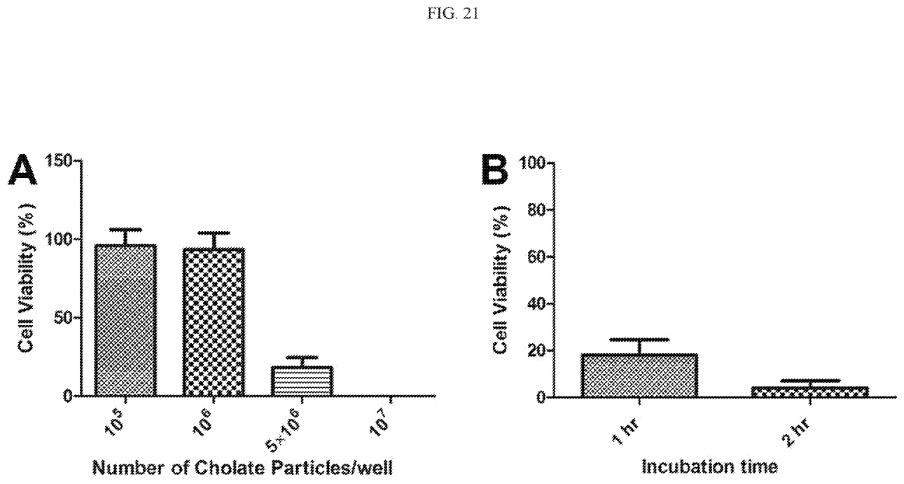

Brightfield microscopy images of the HT-116 cancer cells after being incubated with various concentrations of cholate composite microparticles are shown in FIG. 20. Above a threshold concentration, incubation with the composite microparticles resulted in the detachment of the cell from the plate and their aggregation, which are all indicators of apoptosis. The same kind of results were confirmed once the viability of the cells after being incubated with the cholate composite microparticles was quantified. Incubation of the cells with 5×10⁶ per well of a 24-well plates composite microparticles resulted in the death of nearly 80% of the cells in one hour (FIG. 21A). The cell death was increased to nearly 100% when the concentration of the particles was 2-times increased to 10⁷ particles/well (FIG. 21A). Quantification of the viability of the cells after being incubated with the particles for different time-points confirmed that the apoptotic effect of the salts is time-dependent. While incubation of the cells with 5×10⁶ per well for 1 h induced apoptosis in nearly 80% of the cells, this number was increased to around 95% when the incubation time was increased to 2 h (FIG. 21B).

### Example 10

### Preparation of Silver-Templated Cholate Particles

30 mg silver nitrate was dissolved in 50 microliters water and briefly vortexed for 5 seconds to dissolve completely. The 50 microliters of silver solution was then pipetted into 1 mL ethyl acetate and was emulsified via vortexing for 10 seconds. 2 milliliters of 1 wt % sodium cholate solution was immediately added to the first emulsion and emulsified via vortexing for 10 seconds. The emulsion initially formed into a gel, and then returned to liquid phase as mixing continued. This double emulsion was then added to 10 milliliters of 0.3 wt % sodium cholate solution and mixed via vortexing for 10 seconds. The final emulsion was then placed in a water bath set to 80° C. for 60 minutes. The solution was then removed from the bath and placed on a stir plate to mix at 300 rpm. Rod shaped particles had formed after stirring for 1 hour. The stirred solution containing particles was then centrifuged at 1000 rpm for 5 minutes to isolate particles in the form of a pellet. Supernatant was removed and discarded. Particles were resuspended in water and lyophilized for 24 hours for long term storage.

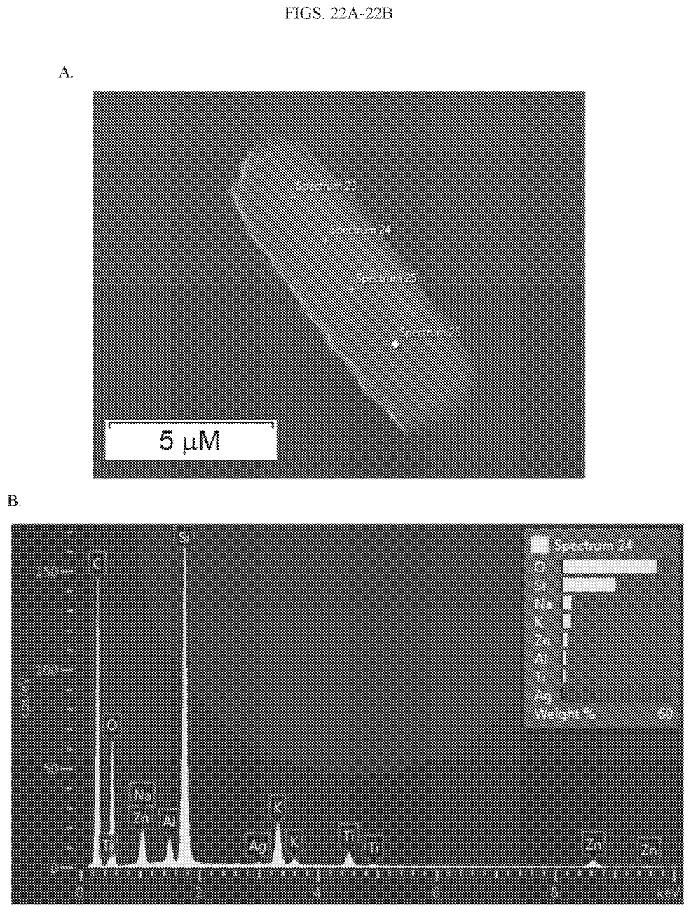

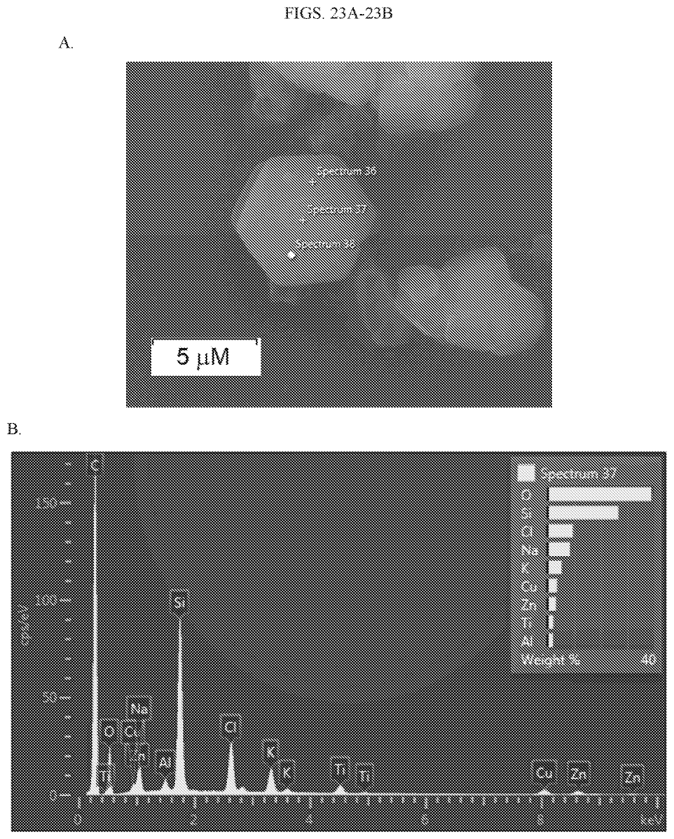

An SEM image of a composite particle produced by this method is shown in FIG. 22A, with an EDS spectrum shown in FIG. 22B. The EDS readouts require a higher beam strength to get adequate readings, and the glass slide beneath the sample accounts for the Si, Al, Na, Zn, K, and Ti peaks. Nevertheless, the particles include the cholate salt as evidenced by the C and O peaks, and the presence of the Ag peak confirms that the particles include the silver nanoparticle templates.

### Example 11

### Preparation of Copper-Templated Cholate Particles

25 mg copper (II) chloride was dissolved in 50 microliters water and briefly vortexed for 5 seconds to dissolve completely. The 50 microliters of copper solution was then pipetted into 1 mL ethyl acetate and was emulsified via vortexing for 30 seconds. 2 milliliters of 1 wt % sodium cholate solution was immediately added to the first emulsion and emulsified via vortexing for 30 seconds. This double emulsion was then added to 10 milliliters of 0.3 wt % sodium cholate solution and mixed via vortexing for 10 seconds. The final emulsion was then placed in a water bath set to 80° C. for 60 minutes. The emulsion was briefly removed and mixed for approximately 5 seconds, and then placed in the 80° C. bath for another 30 minutes. The solution was then removed from the bath, filtered to remove any large precipitates greater than 50 microns in diameter, and placed on a stir plate to mix at 300 rpm. Hexagons are present in solution almost immediately after stirring begins (<30 seconds). The solution can then be rapidly cooled to room temperature via an ice water bath. The filtered and stirred solution containing particles was then centrifuged at 500 rpm for 5 minutes to isolate particles in the form of a pellet. Supernatant was removed and discarded. Particles were resuspended in water and lyophilized for 24 hours for long term storage.

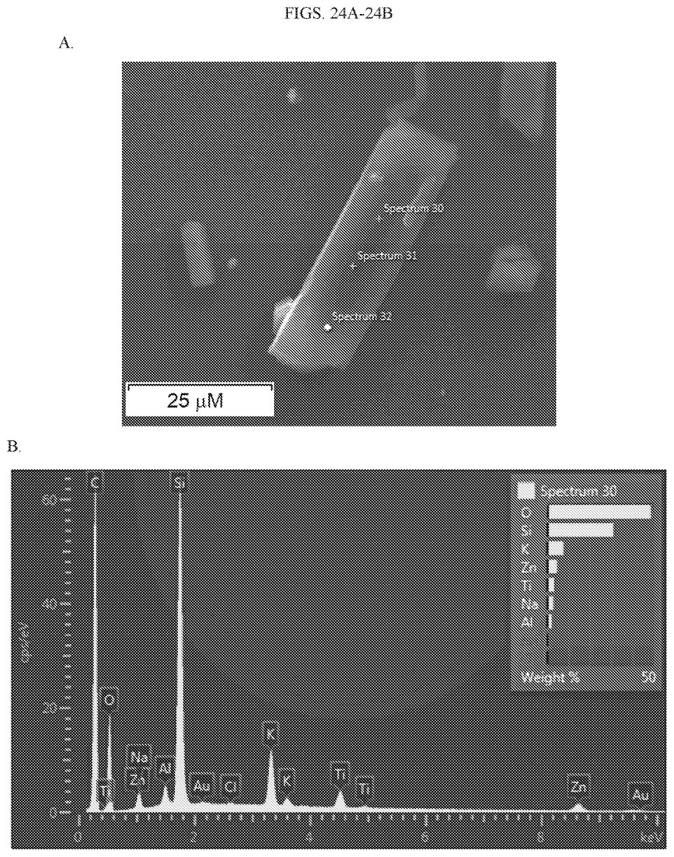

An SEM image of a composite particle produced by this method is shown in FIG. 23A, with an EDS spectrum shown in FIG. 23B. The EDS readouts require a higher beam strength to get adequate readings, and the glass slide beneath the sample accounts for the Si, Al, Na, Zn, K, and Ti peaks. Nevertheless, the particles include the cholate salt as evidenced by the C and O peaks, and the presence of the Cu peak confirms that the particles include the copper nanoparticle templates.

### Example 12

### Preparation of Methylprednisolone Particles

Methylprednisolone particles were prepared in the same manner as the gold-templated cholate particles described in Example 1, with methylprednisolone succinate replacing the sodium cholate.

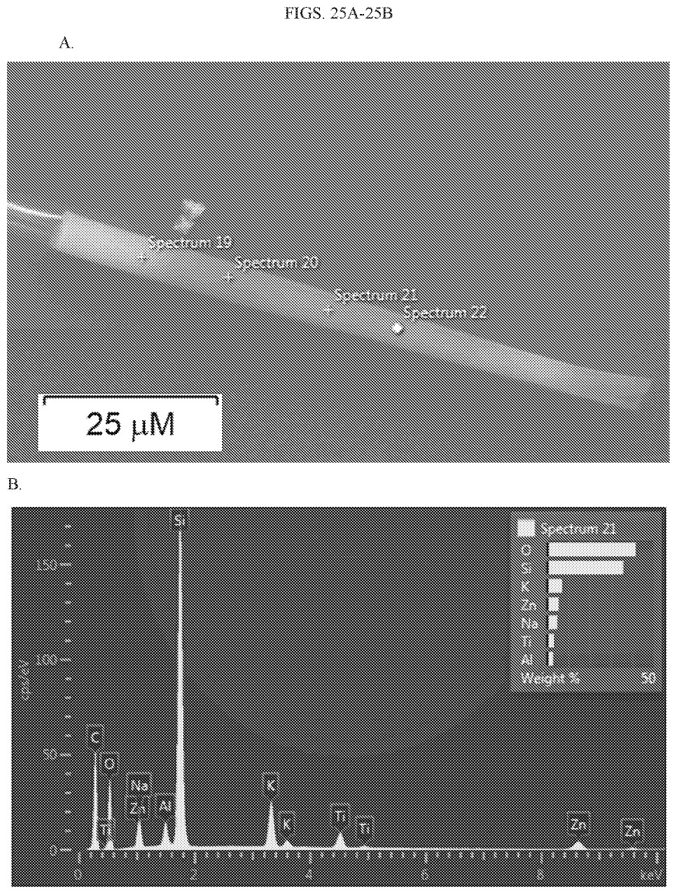

An SEM image of a composite particle produced by this method is shown in FIG. 24A, with an EDS spectrum shown in FIG. 24B. The EDS readouts require a higher beam strength to get adequate readings, and the glass slide beneath the sample accounts for the Si, Al, Na, Zn, K, and Ti peaks. Nevertheless, the particles include the methylprednisolone as evidenced by the C and O peaks, and the presence of the Au peak confirms that the particles include the gold nanoparticle templates.

### Example 13

### Preparation of Hydrocortisone Particles

Hydrocortisone particles were prepared in the same manner as the gold-templated cholate particles described in Example 1, with hydrocortisone succinate replacing sodium cholate, and the heating step increased to 25 minutes at 45° C.

An SEM image of a composite particle produced by this method is shown in FIG. 25A, with an EDS spectrum shown in FIG. 25B. The EDS readouts require a higher beam strength to get adequate readings, and the glass slide beneath the sample accounts for the Si, Al, Na, Zn, K, and Ti peaks. Nevertheless, the particles include the hydrocortisone as evidenced by the C and O peaks, and the presence of the Au peak confirms that the particles include the gold nanoparticle templates.

### Example 14

### Particle Images Under Brightfield and Polarized Light

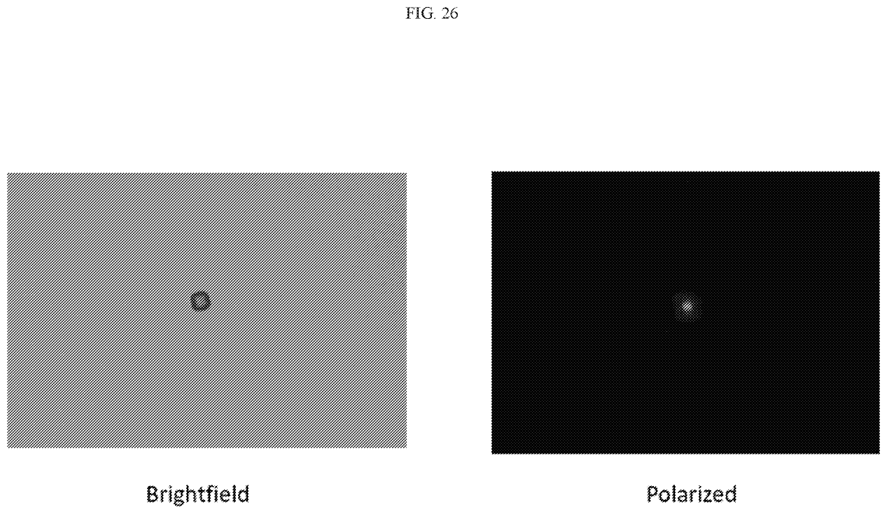

A gold-templated sodium cholate particle was imaged under a brightfield lens and a polarized lens. Images are shown in FIG. 26 (images taken at 40× magnification). A clearly defined diffraction pattern would be consistent with crystalline structures. The polarized lens image suggests that the internal structure of these particles is amorphous due to absence of any diffraction pattern.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A composite particle comprising:
(i) a compound having a steroid core structure, or a salt or ester thereof; and
(ii) transition metal nanoparticles.

2. The composite particle of claim 1, wherein the compound having a steroid core structure is selected from the group consisting of testosterone, exemestane, formestane, mesterolone, fluoxymesterone, methyltestosterone, oxandrolone, oxymetholone, mestranol, norethindrone, danazol, gestrinone, levonorgestrel, lynestrenol, norgestrel, desogestrel, etonogestrel, tibolone, ethynodiol, cyproterone, megestrol, abiraterone, dienogest, mifepristone, drospirenone, spironolactone, estradiol, polyestradiol, estramustine, estrone, estropipate, progesterone, dydrogesterone, hydroxyprogesterone, medroxyprogesterone, segesterone, norelgestromin, norgestimate, cortisol, cortisone, fluorometholone, difluprednate, fludrocortisone, fluocinolone, loteprednol, methylprednisolone, prednicarbate, prednisolone, prednisone, triamcinolone, alclometasone, betamethasone, clobetasol, clobetasone, clocortolone, desoximetasone, dexamethasone, diflorasone, difluocortolone, fluticasone, halometasone, mometasone, rimexolone, amcinonide, budesonide, ciclesonide, deflazacort, desonide, flunisolide, fluocinonide, halcinonide, cholesterol, estradiol, hydrocortisone, diflucortolone, boldenone, nandrolone, altrenogest, stanozolol, osaterone, estriol, aglepristone, trilostane, flumethasone, deoxycorticosterone, alfaxalone, desoxycorticosterone, and isoflupredone, or a salt or an ester thereof, or any combination thereof.

3. The composite particle of claim 1, wherein the compound having a steroid core structure is a bile acid.

4. The composite particle of claim 3, wherein the bile acid is selected from cholic acid, deoxycholic acid, chenodeoxycholic acid, lithocholic acid, glycocholic acid, taurocholic acid, glycodeoxycholic acid, taurodeoxycholic acid, glycochenodeoxycholic acid,
taurochenodeoxycholic acid, glycolithocholic acid, taurolithocholic acid, ursodeoxycholic acid, glycoursodeoxycholic acid, tauroursodeoxycholic acid, and obeticholic acid.

5. The composite particle of claim 3, wherein the bile acid is selected from cholic acid, deoxycholic acid, ursodeoxycholic acid, and chenodeoxycholic acid.

6. The composite particle of claim 1, wherein compound having a steroid core structure is a salt of a bile acid.

7. The composite particle of claim 6, wherein the salt of the bile acid is selected from sodium cholate, sodium deoxycholate, sodium ursodeoxycholate, and sodium chenodeoxycholate.

8. The composite particle of claim 1, wherein the compound having a steroid core structure is a corticosteroid compound.

9. The composite particle of claim 8, wherein the corticosteroid compound is selected from hydrocortisone, dexamethasone, beclomethasone, ciclesonide, clobetasol, clobetasone, desonide, desoxymethasone, desoxycorticosterone, dichlorisone, diflorasone, diflucortolone, fluclarolone, fludrocortisone, flumethasone, fluocinolone, fluocinonide, flucortine, fluocortolone, fluprednidene, flurandrenolone, halcinonide, halometasone, methylprednisolone, triamcinolone, cortisone, cortodoxone, flucetonide, fluradrenalone, medrysone, alclometasone, amciafel, amcinafide, amcinonide, betamethasone, budesonide, chlorprednisone, clocortelone, clescinolone, difluprednate, flucloronide, flunisolide, fluoromethalone, fluperolone, fluprednisolone, hydrocortamate, meprednisone, mometasone, paramethasone, prednisolone, prednisone, prednicarbate, and tixocortol, or a salt or an ester thereof.

10. The composite particle of claim 9, wherein the corticosteroid compound is selected from methylprednisolone and hydrocortisone, or a salt or an ester thereof.

11. The composite particle of any one of claims 1-10, wherein the particle has a hexagonal prism shape.

12. The composite particle of claim 11, wherein the hexagonal prism has a diagonal length of 2.5 μm to 10 μm.

13. The composite particle of claim 11 or claim 12, wherein the hexagonal prism has a height of 2.5 μm to 6.5 μm.

14. The composite particle of any one of claims 1-10, wherein the particle has a rod shape.

15. The composite particle of claim 14, wherein the rod has a length of 2.5 μm to 100 μm.

16. The composite particle of claim 14 or claim 15, wherein the rod has a length of 10 μm to 50 μm.

17. The composite particle of any one of claims 1-10, wherein the particle has a hexagonal sheet shape.

18. The composite particle of claim 17 wherein the hexagonal sheet has a long side length of 10 μm to 50 μm, and a short side length of 5 μm to 20 μm.

19. The composite particle of any one of claims 1-10, wherein the particle has a spherical shape.

20. The composite particle of claim 19, wherein the sphere has a diameter of 1 μm to 10 μm.

21. The composite particle of any one of claims 1-20, wherein the transition metal nanoparticles are gold, silver, copper, platinum, palladium, nickel, or iron nanoparticles.

22. The composite particle of claim 21, wherein the transition metal nanoparticles are gold, silver, or copper nanoparticles.

23. The composite particle of claim 21, wherein the transition metal nanoparticles are gold nanoparticles.

24. The composite particle of claim 21, wherein the transition metal nanoparticles are silver nanoparticles.

25. The composite particle of claim 21, wherein the transition metal nanoparticles are copper nanoparticles.

26. The composite particle of any one of claims 1-25, wherein the particle consists essentially of the compound having a steroid core structure or salt or ester thereof, and the transition metal nanoparticles.

27. A composition comprising a plurality of composite particles of any one of claims 1-26.

28. The composition of claim 27, further comprising a pharmaceutically acceptable carrier.

29. A method of making a plurality of composite particles of any one of claims 1-2632, comprising:
(a) providing a first solution of a transition metal salt in water;
(b) adding a hydrophobic solvent to the solution and mixing to form a first emulsion;
(c) combining the first emulsion and a second solution, wherein the second solution comprises a compound having a steroid core structure, or a salt or ester thereof, and mixing to form a second emulsion;
(d) combining the second emulsion and a third solution, wherein the third solution comprises the compound having a steroid core structure or a salt or ester thereof, to form a final mixture; and
(e) incubating the final mixture to form the plurality of composite particles.

30. The method of claim 29, wherein the second and third solutions comprise a salt of a bile acid.

31. The method of claim 30, wherein the salt of the bile acid is selected from sodium cholate, sodium deoxycholate, sodium ursodeoxycholate, and sodium chenodeoxycholate.

32. The method of claim 29, wherein the second and third solutions comprise a corticosteroid compound.

33. The method of claim 32, wherein the corticosteroid compound is selected from methylprednisolone and hydrocortisone, or an ester thereof.

34. The method of any one of claims 29-33, wherein the first solution comprises a gold(III) salt, a silver(I) salt, a copper(II) salt, a platinum(II) salt, a palladium(II) salt, a nickel(II) salt, an iron(II) salt, or an iron(III) salt.

35. The method of any one of claims 29-34, wherein the first solution comprises a gold(III) salt, a silver(I) salt, or a copper(II) salt.

36. The method of claim 35, wherein the first solution comprises HAuCl₄.

37. The method of claim 29, wherein the compound having a steroid core structure is a bile acid or a salt or ester thereof, the metal salt is HAuCl₄, and the HAuCl₄ and the bile acid or salt or ester thereof are present in the final mixture in a mass ratio of at least 0.2.

38. The method of any one of claims 29-37, wherein the incubation step (e) comprises heating the final mixture at a temperature of 40° C. to 100° C. for 10 minutes to 120 minutes.

39. The method of any one of claims 29-38, wherein the incubation step (e) comprises heating the final mixture at 45° C. for 15 minutes.

40. The method of any one of claims 29-39, further comprising removing the solvent from the final mixture after the incubating step.

41. The method of any one of claims 29-40, wherein the hydrophobic solvent is ethyl acetate or dichloromethane.

42. The method of any one of claims 29-41, further comprising separating the composite particles from the final mixture.

43. A method of treating a liver disease or a peroxisomal disorder in a subject in need of treatment, comprising administering to the subject a therapeutically effective amount of a composition of claim 27 or claim 28.

44. The method of claim 43, wherein the liver disease is a bile acid synthesis disorder or primary biliary cholangitis.

45. The method of claim 43, wherein the liver disease is a bile acid synthesis disorder due to a single enzyme defect.

46. The method of claim 43, wherein the peroxisomal disorder is a Zellweger spectrum disorder.

47. A method of non-surgical removal of a localized fat deposit in a subject, comprising contacting the deposit with an effective amount of a composition of claim 27 or claim 28.

48. A method of reducing a subcutaneous fat deposit in a subject in need thereof, comprising administering locally to the subcutaneous fat deposit in the subject an effective amount of a composition of claim 27 or claim 28.

49. A method of treating cancer in a subject in need thereof, comprising administering to the subject a therapeutically effective amount of a composition of claim 27 or claim 28.

50. The method of claim 29, wherein the cancer is selected from colorectal cancer and gastric cancer.

51. A method of reducing the proliferation of cancer cells, comprising contacting the cells with an effective amount of a composition of claim 27 or claim 28.

52. The method of claim 51, wherein the cancer cells are selected from colorectal cancer cells and gastric cancer cells.

53. A method of treating a disorder selected from the group consisting of endocrine disorders, rheumatic disorders, collagen diseases, dermatologic diseases, allergic states, ophthalmic diseases, respiratory diseases, hematologic disorders, neoplastic diseases, gastrointestinal diseases, nervous system disorders, inflammatory disorders, and renal diseases, comprising administering to the subject a therapeutically effective amount of a composition of claim 27 or claim 28.

54. Use of a particle or composition of any of claims 1-28.

55. Use of a particle or composition of any of claims 1-28 for removal of a localized fat deposit.

56. Use of a particle or composition of any of claims 1-28 for treating a disease selected from liver diseases, a peroxisomal disorder, cancer, endocrine disorders, rheumatic disorders, collagen diseases, dermatologic diseases, allergic states, ophthalmic diseases, respiratory diseases, hematologic disorders, neoplastic diseases, gastrointestinal diseases, nervous system disorders, inflammatory disorders, and renal diseases.
